(19) United States
(12) Patent Application Publication
(10) Pub. No.: US 2024/0066293 A1
(43) Pub. Date: Feb. 29, 2024

(54) SPINAL CORD STIMULATION DEVICE IMPLANTATION METHODS AND RELATED SYSTEMS AND DEVICES

(71) Applicant: NeuroOne Medical Technologies Corporation, Eden Prairie, MN (US)

(72) Inventors: Maria Francisca Porto Cruz Westermann, Düsseldorf (DE); Maria Vomero, Astoria, NY (US); Camilo Diaz-Botia, Monte Sereno, CA (US); Samuel Ong, San Francisco, CA (US); Alfonso Chavez, San Jose, CA (US); William Walter Norman, Saint Anthony, MN (US); Marcus Allen Szamlewski, Eden Prairie, MN (US)

(21) Appl. No.: 18/455,258

(22) Filed: Jun. 21, 2023

Related U.S. Application Data
(60) Provisional application No. 63/400,428, filed on Aug. 24, 2022.

(51) Int. Cl. A61N 1/05; A61B 17/34

(52) U.S. Cl. CPC A61N 1/0558; A61B 17/3468

(57) ABSTRACT

Methods and devices for implanting spinal cord stimulation devices, including thin-film spinal cord stimulation devices. In some embodiments, the devices include an introduction needle and/or sheath, a stylet, and a pushing device for urging a stimulation device into the epidural space by controlling the proximal end of the device, including, in some cases, deformable thin-film spinal cord stimulation devices. Further implementations include a deployment device for ensuring deployment of the stimulation device in the epidural space. In other embodiments, the devices include sutures attached to a distal portion of the stimulation device, at least one introduction tube, a lead introduction device, and/or a suture introduction device for urging a stimulation device into the epidural space by controlling both the proximal and distal ends of the device, including, in some cases, deformable thin-film spinal cord stimulation devices.

### CROSS-REFERENCE TO RELATED APPLICATION(S)

This application claims the benefit under 35 U.S.C. § 119(e) to U.S. Provisional Application 63/400,428, filed Aug. 24, 2022 and entitled “Spinal Cord Stimulation Device Implantation Methods and Related Systems and Devices,” which is hereby incorporated herein by reference in its entirety.

### FIELD

The various embodiments herein relate to methods of implanting stimulation devices for stimulating the spinal cord and/or peripheral nerves and related systems and devices.

### BACKGROUND

Electrical stimulation of the spinal cord can result in pain reduction and/or elimination. Medical devices having electrodes (also referred to as “stimulators” or “leads”) are often implanted near the spinal column to provide pain relief for chronic intractable pain. The electrodes stimulate tissue within the spinal column to reduce pain sensations at other parts of the body. The stimulation signals applied can be optimized for pain reduction or elimination depending on the location of the pain.

Thin film spinal cord stimulation devices can be difficult to implant using standard methods. For example, there is little control of the distal portions of the device when positioning the device in the patient. More specifically, when urging the device distally via the tail of the device, the force may not be transmitted to the distal portions of the device because the device will tend to buckle near its tail. Further, precise placement of a thin film device can be challenging. That is, when retracting the casing/tool that carries the device into the epidural space, the device might be also retracted or displaced. In addition, alignment of the device can be difficult. That is, a thin film device can tend to slip or curve while it is being urged into the epidural space.

There is a need in the art for improved methods of implanting thin film spinal cord stimulation devices and related systems and devices.

### BRIEF SUMMARY

Discussed herein are various devices and methods for implanting a spinal cord stimulation device into a patient, including, in some embodiments, a deformable thin-film spinal cord stimulation device.

In Example 1, a thin film spinal cord stimulation system comprises a spinal cord stimulation device comprising an elongate thin film lead body and a thin film electrode body disposed at one end of the elongate thin film lead body, the thin film electrode body comprising at least two contacts disposed on the electrode body and a pusher receiving structure disposed on a distal portion of the thin film electrode body. The system further comprises elongate pusher device comprising an elongate pusher body and a distal tip disposed at a distal end of the elongate pusher body, wherein the distal tip is constructed and arranged to be coupleable with the pusher receiving structure.

Example 2 relates to the thin film spinal cord stimulation system according to Example 1, further comprising an introduction needle comprising a lumen sized to receive the spinal cord stimulation device and the elongate pusher device therethrough.

Example 3 relates to the thin film spinal cord stimulation system according to Example 2, further comprising a steerable stylet configured to be positionable through the introduction needle.

Example 4 relates to the thin film spinal cord stimulation system according to Example 2, further comprising an introduction sheath comprising a lumen having a larger inner diameter than the lumen of the introduction needle.

Example 5 relates to the thin film spinal cord stimulation system according to Example 4, further comprising a funnel comprising a lumen and a distal end, wherein the distal end is coupleable to a proximal end of the introduction sheath.

Example 6 relates to the thin film spinal cord stimulation system according to Example 1, wherein the pusher receiving structure is a collar comprising a lumen.

Example 7 relates to the thin film spinal cord stimulation system according to Example 1, further comprising a deployment device comprising an elongate deployment device body and an expandable structure disposed at a distal end of the elongate deployment device body, the expandable structure configured to move between a retracted configuration and an expanded configuration.

Example 8 relates to the thin film spinal cord stimulation system according to Example 7, wherein the expandable structure comprises two expandable wings or an expandable sleeve.

In Example 9, a method of implanting a thin film spinal cord stimulation device comprises inserting an introduction needle between two spinous processes and into an epidural space of a patient, inserting a steerable stylet through the introduction needle such that the guidewire extends distally from the introduction needle within the epidural space, inserting the thin film spinal cord stimulation device over the steerable stylet, inserting a pusher device over the steerable stylet and into engaging contact with the thin film spinal cord stimulation device, urging the thin film spinal cord stimulation device through the introduction needle by urging the pusher device distally over the steerable stylet such that the thin film spinal cord stimulation device is disposed in the epidural space, and removing the pusher device, steerable stylet, and introduction needle.

Example 10 relates to the method according to Example 9, wherein the thin film spinal cord stimulation device comprises an elongate thin film lead body and a thin film electrode body disposed at one end of the elongate thin film lead body, the thin film electrode body comprising at least two contacts disposed on the electrode body and a pusher receiving structure disposed on a distal portion of the thin film electrode body.

Example 11 relates to the method according to Example 10, wherein the inserting the pusher device into engaging contact with the thin film spinal cord stimulation device comprises urging the pusher device into contact with the pusher receiving structure such that the pusher device is coupled with the pusher receiving structure.

Example 12 relates to the method according to Example 9, wherein the removing the introduction needle comprises removing the introduction needle after inserting the steerable stylet through the introduction needle.

Example 13 relates to the method according to Example 12, further comprising inserting an introduction sheath over the steerable stylet after removing the introduction needle such that the introduction sheath is inserted between the two spinous processes and into the epidural space.

Example 14 relates to the method according to Example 13, further comprising inserting a funnel over the steerable stylet and coupling the funnel to the introduction sheath.

Example 15 relates to the method according to Example 13, further comprising inserting a deployment device through the introduction sheath and retracting the deployment device to assist with the deployment of the thin film spinal cord stimulation device.

In Example 16, a method of implanting a thin film spinal cord stimulation device comprises inserting an introduction needle between two spinous processes and into an epidural space of a patient inserting a steerable stylet through the introduction needle such that the guidewire extends distally from the introduction needle within the epidural space, and inserting the thin film spinal cord stimulation device over the steerable stylet, wherein the thin film spinal cord stimulation device comprises an elongate thin film lead body and a thin film electrode body disposed at one end of the elongate thin film lead body, the thin film electrode body comprising at least two contacts disposed on the electrode body and a pusher receiving structure disposed on a distal portion of the thin film electrode body. The method further comprises inserting a pusher device over the steerable stylet and coupling the pusher device with the pusher receiving structure, urging the thin film spinal cord stimulation device through the introduction needle by urging the pusher device distally over the steerable stylet such that the thin film spinal cord stimulation device is disposed in the epidural space, and removing the pusher device, steerable stylet, and introduction needle.

Example 17 relates to the method according to Example 16, wherein the inserting the thin film spinal cord stimulation device over the steerable stylet comprises inserting a proximal end of the steerable stylet through the pusher receiving structure.

Example 18 relates to the method according to Example 16, further comprising removing the introduction needle after inserting the steerable stylet through the introduction needle and inserting an introduction sheath over the steerable stylet such that the introduction sheath is inserted between the two spinous processes and into the epidural space.

Example 19 relates to the method according to Example 18, further comprising inserting a funnel over the steerable stylet and coupling the funnel to the introduction sheath.

Example 20 relates to the method according to Example 18, further comprising inserting a deployment device through the introduction sheath and retracting the deployment device to assist with the deployment of the thin film spinal cord stimulation device.

In Example 21, a method of implanting a spinal cord stimulation device comprises inserting distal and proximal tubes into an epidural space of a patient, inserting a guidewire through the distal and proximal tubes such that the guidewire is disposed through the distal and proximal tubes and the epidural space disposed between the distal and proximal tubes, inserting a lead introduction sheath through the proximal tube such that a distal end of the lead introduction sheath is disposed within the epidural space, inserting a suture introduction device through the distal and proximal tubes such that the suture introduction device is disposed through the distal and proximal tubes, the lead introduction sheath, and the epidural space disposed between the distal and proximal tubes, attaching sutures to a first end of the suture introduction device at a first end of the sutures, wherein a stimulation device is attached to a second end of the sutures, pulling the second end of suture introduction device such that the sutures are urged through and disposed within the distal and proximal tubes, the lead introduction sheath, and the epidural space, detaching the suture introduction device from the first end of the sutures, pulling the first end of the sutures such that a distal end of the stimulation device is urged through the lead introduction sheath and into the epidural space, and positioning the stimulation device via the sutures and a proximal lead of the stimulation device.

Example 22 relates to the method according to Example 21, wherein the stimulation device attached to the second of the sutures is disposed within a device delivery case.

In Example 23, a method of implanting a spinal cord stimulation device comprises inserting a distal tube into an epidural space of a patient, inserting a guidewire through the distal tube and an opening formed as a result of a laminotomy such that the guidewire is disposed through the distal tube, the opening, and the epidural space disposed between the distal tube and the opening, inserting a suture introduction device through the distal tube and the opening such that the suture introduction device is disposed through the distal tube, the opening, and the epidural space disposed between the distal tube and the opening, attaching sutures to a first end of the suture introduction device at a first end of the sutures, wherein a stimulation device is attached to a second end of the sutures, pulling the second end of suture introduction device such that the sutures are urged through and disposed within the distal tube, the opening, and the epidural space, detaching the suture introduction device from the first end of the sutures, pulling the first end of the sutures such that a distal end of the stimulation device is urged into the epidural space, and positioning the stimulation device via the sutures and a proximal lead of the stimulation device.

Example 24 relates to the method according to Example 23, further comprising optionally dilating the epidural space after inserting the guidewire.

Example 25 relates to the method according to Example 23, wherein the stimulation device attached to the second of the sutures is disposed within a device delivery case.

Example 26 relates to the method according to Example 23, further comprising removing the device delivery case, whereby a deployment device disposed adjacent to the stimulation device begins to urge a paddle of the stimulation device to expand toward a deployed configuration.

While multiple embodiments are disclosed, still other embodiments will become apparent to those skilled in the art from the following detailed description, which shows and describes illustrative embodiments. As will be realized, the various implementations are capable of modifications in various obvious aspects, all without departing from the spirit and scope thereof. Accordingly, the drawings and detailed description are to be regarded as illustrative in nature and not restrictive.

### BRIEF DESCRIPTION OF THE DRAWINGS

FIG. 1A is a top view of a percutaneous spinal cord stimulation device, according to one embodiment.

FIG. 1B is a top view of a paddle spinal cord stimulation device, according to one embodiment.

FIG. 2A is a top view of a spinal cord stimulation device disposed over a stylet, according to one embodiment.

FIG. 2B is a side view of the spinal cord stimulation device of FIG. 2A, according to one embodiment.

FIG. 3A is a top view of a spinal cord stimulation device and a pusher device disposed over a stylet, according to one embodiment.

FIG. 3B is a side view of the spinal cord stimulation device and pusher device of FIG. 3A, according to one embodiment.

FIG. 3C is a top view of the pusher device engaged with the spinal cord stimulation device of FIG. 3A, according to one embodiment.

FIG. 3D is a side view of the spinal cord stimulation device and pusher device of FIG. 3C, according to one embodiment.

FIG. 4 is a top view of an introduction sheath disposed over a stylet, according to one embodiment.

FIG. 5 is a top view of a funnel attached to an introduction sheath disposed over a stylet, according to one embodiment.

FIG. 6A is a top view of a funnel attached to an introduction sheath and a pusher device engaged with a spinal cord stimulation device disposed over a stylet, according to one embodiment.

FIG. 6B is a side view of the funnel attached to the introduction sheath and the pusher device engaged with the spinal cord stimulation device of FIG. 6A, according to one embodiment.

FIG. 6C is a top view of the spinal cord stimulation device of FIG. 6A being inserted into the funnel, according to one embodiment.

FIG. 6D is a side view of the spinal cord stimulation device of FIG. 6C being inserted into the funnel, according to one embodiment.

FIG. 6E is a top view of the spinal cord stimulation device of FIG. 6A being inserted into the sheath, according to one embodiment.

FIG. 6F is a side view of the spinal cord stimulation device of FIG. 6E being inserted into the sheath, according to one embodiment.

FIG. 7A is a schematic cross-sectional view of an introduction needle being inserted into the epidural space of a patient, according to one embodiment.

FIG. 7B is a schematic cross-sectional view of a stylet inserted through the introduction needle of FIG. 7A, according to one embodiment.

FIG. 7C is a schematic cross-sectional view of an spinal cord stimulation device being urged over the stylet of FIG. 7B, according to one embodiment.

FIG. 7D is a top view of the spinal cord stimulation device of FIG. 7C, according to one embodiment.

FIG. 7E is a side view of the spinal cord stimulation device of FIG. 7D, according to one embodiment.

FIG. 7F is a schematic cross-sectional view of a pusher device engaged with the spinal cord stimulation device of FIG. 7C, according to one embodiment.

FIG. 7G is a top view of the spinal cord stimulation device and pusher device of FIG. 7F, according to one embodiment.

FIG. 7H is a side view of the spinal cord stimulation device and pusher device of FIG. 7F, according to one embodiment.

FIG. 7I is a schematic cross-sectional view of the pusher device and the spinal cord stimulation device of FIG. 7F being urged into the introduction needle, according to one embodiment.

FIG. 7J is a schematic cross-sectional view of the pusher device and the spinal cord stimulation device of FIG. 7F being urged through the introduction needle and into the epidural space, according to one embodiment.

FIG. 7K is a schematic cross-sectional view of the spinal cord stimulation device of FIG. 7J with the pusher device, the stylet, and the introduction needle removed, according to one embodiment.

FIG. 8A is a schematic cross-sectional view of an introduction needle being inserted into the epidural space of a patient, according to one embodiment.

FIG. 8B is a schematic cross-sectional view of a stylet inserted through the introduction needle of FIG. 8A, according to one embodiment.

FIG. 8C is a schematic cross-sectional view of an spinal cord stimulation device being urged over the stylet of FIG. 8B, according to one embodiment.

FIG. 8D is a schematic cross-sectional view of a pusher device engaged with the spinal cord stimulation device of FIG. 8C, according to one embodiment.

FIG. 8E is a schematic cross-sectional view of the pusher device and the spinal cord stimulation device of FIG. 8D being urged into the introduction needle, according to one embodiment.

FIG. 8F is a schematic cross-sectional view of the pusher device and the spinal cord stimulation device of FIG. 8E being urged through the introduction needle and into the epidural space, according to one embodiment.

FIG. 8G is a schematic cross-sectional view of the spinal cord stimulation device of FIG. 8F with the pusher device, the stylet, and the introduction needle removed, according to one embodiment.

FIG. 9A is a schematic cross-sectional view of an introduction needle being inserted into the epidural space of a patient, according to one embodiment.

FIG. 9B is a schematic cross-sectional view of a stylet inserted through the introduction needle of FIG. 9A, according to one embodiment.

FIG. 9C is a schematic cross-sectional view of the stylet of FIG. 9B with the introduction needle removed, according to one embodiment.

FIG. 9D is a schematic cross-sectional view of an introduction sheath inserted over the stylet of FIG. 9C, according to one embodiment.

FIG. 9E is a schematic cross-sectional view of an spinal cord stimulation device being urged over the stylet of FIG. 9D, according to one embodiment.

FIG. 9F is a schematic cross-sectional view of a pusher device engaged with the spinal cord stimulation device of FIG. 9E, according to one embodiment.

FIG. 9G is a schematic cross-sectional view of the pusher device and the spinal cord stimulation device of FIG. 9F being urged into the introduction sheath, according to one embodiment.

FIG. 9H is a schematic cross-sectional view of the pusher device and the spinal cord stimulation device of FIG. 9G being urged through the introduction needle and into the epidural space, according to one embodiment.

FIG. 9I is a schematic cross-sectional view of the spinal cord stimulation device of FIG. 9H with the pusher device, the stylet, and the introduction sheath removed, according to one embodiment.

FIG. 10A is a top view of a deployment device and a spinal cord stimulation device disposed through an introduction needle or sheath, according to one embodiment.

FIG. 10B is a side view of the deployment device and spinal cord stimulation device of FIG. 10A disposed through introduction needle or sheath, according to one embodiment.

FIG. 10C is a top view of the deployment device and the spinal cord stimulation device of FIG. 10A being urged out of the introduction needle or sheath, according to one embodiment.

FIG. 10D is a side view of the deployment device and spinal cord stimulation device of FIG. 10C being urged out of the introduction needle or sheath, according to one embodiment.

FIG. 10E is a top view of the deployment device and the spinal cord stimulation device of FIG. 10C after being urged out of the introduction needle or sheath, according to one embodiment.

FIG. 10F is a side view of the deployment device and spinal cord stimulation device of FIG. 10E after being urged out of the introduction needle or sheath, according to one embodiment.

FIG. 10G is a top view of the deployment device and the spinal cord stimulation device of FIG. 10E with the deployment device in its deployed position, according to one embodiment.

FIG. 10H is a side view of the deployment device and spinal cord stimulation device of FIG. 10G with the deployment device in its deployed position, according to one embodiment.

FIG. 10I is a top view of the deployment device and the spinal cord stimulation device of FIG. 10G with the deployment device being retracted over the stimulation device to deploy the stimulation device, according to one embodiment.

FIG. 10J is a side view of the deployment device and spinal cord stimulation device of FIG. 10I with the deployment device being retracted over the stimulation device to deploy the stimulation device, according to one embodiment.

FIG. 11A is a side view of a deployment device and spinal cord stimulation device with a pushing device coupled with the stimulation device, according to one embodiment.

FIG. 11B is a top view of the deployment device, spinal cord stimulation device, and pushing device of FIG. 11A, according to one embodiment.

FIG. 11C is a top view of the deployment device, spinal cord stimulation device, and pushing device of FIG. 11A being urged through an introduction needle or sheath, according to one embodiment.

FIG. 11D is a top view of the deployment device, the spinal cord stimulation device, and pushing device of FIG. 11C after being urged out of the introduction needle or sheath, according to one embodiment.

FIG. 11E is a top view of the deployment device, the spinal cord stimulation device, and the pushing device of FIG. 11D with the deployment device being retracted over the stimulation device to deploy the stimulation device, according to one embodiment.

FIG. 11F is a top view of the deployment device, the spinal cord stimulation device, and the pushing device of FIG. 11E with the deployment device being retracted through the introduction needle or sheath, according to one embodiment.

FIG. 12A is a top view of a deployment device, according to another embodiment.

FIG. 12B is an enlarged top view of a distal portion of the deployment device of FIG. 12A, according to one embodiment.

FIG. 12C is an enlarged side view of a distal portion of the deployment device of FIG. 12A in a curled position, according to one embodiment.

FIG. 12D is an enlarged cross-sectional view along the longitudinal axis of the deployment device of FIG. 12A in a curled position, according to another embodiment.

FIG. 13A is a top view of a spinal cord stimulation device with sutures disposed through openings defined in a distal portion of the device, according to one embodiment.

FIG. 13B is a schematic cross-sectional view of the spinal cord stimulation device of FIG. 13A disposed within the epidural space, according to one embodiment.

FIG. 14A is a schematic cross-sectional view of two introduction tubes with trocar needles being inserted into the epidural space of a patient, according to one embodiment.

FIG. 14B is a schematic cross-sectional view of the two introduction tubes of FIG. 14A with the trocar needles removed, according to one embodiment.

FIG. 14C is a schematic cross-sectional view of a guidewire inserted through the introduction tubes of FIG. 14B, according to one embodiment.

FIG. 14D is a schematic cross-sectional view of the guidewire and tubes of FIG. 14C with a lead introduction device inserted over the guidewire and through one of the introduction tubes, according to one embodiment.

FIG. 14E is a schematic cross-sectional view of a suture introduction device inserted over the guidewire and through the other of the introduction tubes of FIG. 14D, according to one embodiment.

FIG. 14F is a schematic cross-sectional view of the suture introduction device and the lead introduction device of FIG. 14E with the guidewire removed, according to one embodiment.

FIG. 14G is a schematic cross-sectional view of the suture introduction device and the lead introduction device of FIG. 14F with sutures attached to the end of the suture introduction device, according to one embodiment.

FIG. 14H is a schematic cross-sectional view of the suture introduction device and the lead introduction device of FIG. 14G in which the suture introduction device is retracted out of the introduction tube such that the sutures are disposed through both introduction tubes, according to one embodiment.

FIG. 14I is a schematic cross-sectional view of the sutures and the lead introduction device of FIG. 14H in which the sutures are urged out of the tube such that the device case with the stimulation device disposed therein is disposed in the epidural space, according to one embodiment.

FIG. 14J is a schematic cross-sectional view of the sutures and the stimulation device of FIG. 14I with the stimulation device disposed in the epidural space with the device case removed, according to one embodiment.

FIG. 14K is a schematic cross-sectional view of the stimulation device of FIG. 14J with the introduction tubes and sutures removed, according to one embodiment.

FIG. 15A is a schematic cross-sectional view of an introduction tube with a trocar needle being inserted into the epidural space of a patient and a spinous process removed to create an opening at a predetermined location, according to one embodiment.

FIG. 15B is a schematic cross-sectional view of the introduction tube of FIG. 15A with the trocar needle removed, according to one embodiment.

FIG. 15C is a schematic cross-sectional view of a guidewire inserted through the introduction tube and the opening of FIG. 15B, according to one embodiment.

FIG. 15D is a schematic cross-sectional view of a dilator being introduced over the guidewire through the opening of FIG. 15C, according to one embodiment.

FIG. 15E is a schematic cross-sectional view of the guidewire and tube of FIG. 15C with a lead introduction device inserted over the guidewire and through the opening and the introduction tube, according to one embodiment.

FIG. 15F is a schematic cross-sectional view of the suture introduction device and the introduction tube of FIG. 15E with the guidewire removed, according to one embodiment.

FIG. 15G is a schematic cross-sectional view of the suture introduction device of FIG. 15F with sutures attached to the end of the suture introduction device, according to one embodiment.

FIG. 15H is a schematic cross-sectional view of the suture introduction device of FIG. 15G in which the suture introduction device is retracted out of the introduction tube such that the sutures are disposed through the opening and the introduction tube and the a device case (containing the stimulation device) is attached to the sutures, according to one embodiment.

FIG. 15I is a schematic cross-sectional view of the sutures and device case of FIG. 15H in which the sutures are urged out of the tube such that the device case with the stimulation device disposed therein is disposed in the epidural space, according to one embodiment.

FIG. 15J is a schematic cross-sectional view of the sutures and the stimulation device of FIG. 15I with the stimulation device disposed in the epidural space with the device case removed, according to one embodiment.

FIG. 15K is a schematic cross-sectional view of the stimulation device of FIG. 15J with the introduction tube and sutures removed, according to one embodiment.

### DETAILED DESCRIPTION

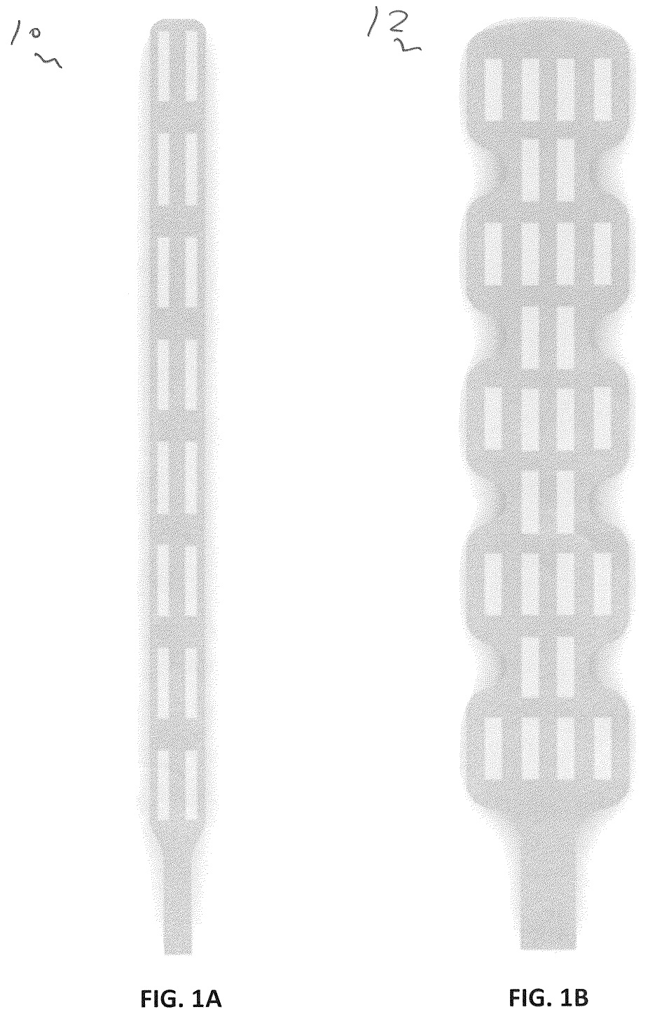

The various embodiments disclosed or contemplated herein relate to improved systems, devices, and methods, and various components thereof, for stimulating the spinal cord or related peripheral nerves in the human body. In certain exemplary implementations, certain stimulation devices that incorporate thin-film technology are provided, along with various implantation methods for implanting those devices. More specifically, methods are provided for implanting both percutaneous spinal cord stimulation devices (also referred to as “linear devices”) such as exemplary percutaneous spinal cord stimulation device 10 as shown in FIG. 1A and paddle spinal cord stimulation devices such as exemplary paddle spinal cord stimulation device 12 as shown in FIG. 1B. In addition, certain thin-film stimulation device implementations are also provided that incorporate structures or features as described in detail herein that can be used to assist with the implantation of those devices, as will be discussed in further detail below. Further, according to some embodiments, additional tools can be used to assist with implantation as well.

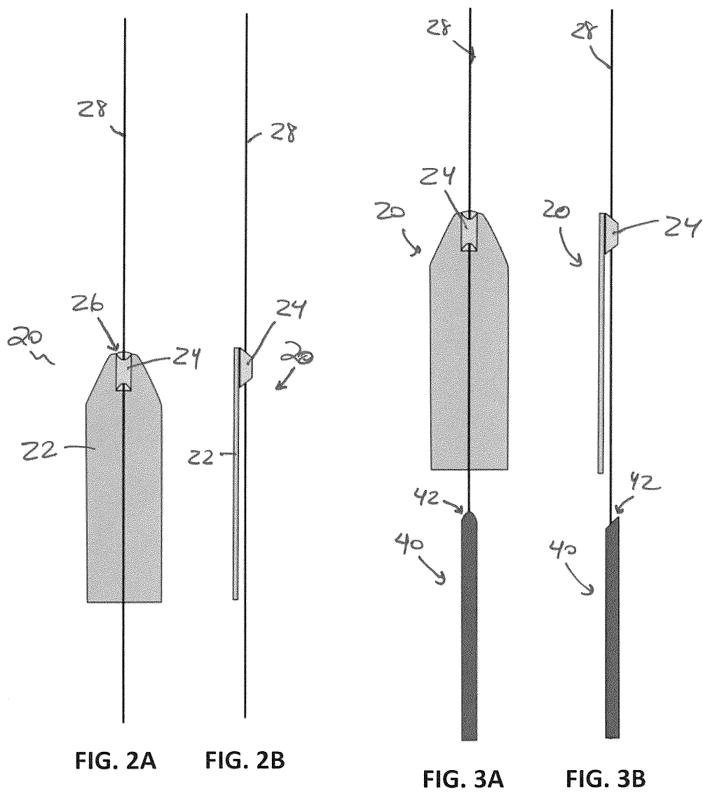

The various stimulation devices disclosed or contemplated herein can include structures or features that assist with the insertion and positioning of those devices in the target area on or near the spinal cord. For example, as shown in FIGS. 2A and 2B, a deformable spinal cord stimulation device 20 is depicted according to one embodiment. The device 20 has an electrode body 22 (also referred to as a “contact body” or “paddle”) on which the one or more electrodes (not shown) are disposed, a lead body (also referred to as a “tail”) (not shown), and a connection component (also referred to as a “proximal connection component,” “connector,” or “proximal connector”) (not shown) to which the external electrical source is coupled. While the device 20 as shown can be a paddle stimulation device 20, it is noted that the device 20 can also be a percutaneous (or “linear”) stimulation device or a hybrid device that has paddle-like structural features but can be implanted percutaneously. In certain implementations, in addition to the specific components and/or features disclosed herein, the device 20 and any of the deformable stimulation devices disclosed or contemplated herein can also have any of the various components and/or features of the deformable stimulation devices disclosed in U.S. patent application Ser. No. 18/358,859, filed on Jul. 25, 2023 and entitled “Deformable Spinal Cord Stimulation Device and Related Systems and Methods,” which is hereby incorporated herein by reference in its entirety.

Each of the components of the device 20 (including the electrode body 22, the lead body, and the proximal connector) can be thin film components, with some of those components or portions of the device 20 being coated in any device embodiments herein with thin layers of silicone rubber (wherein various embodiments of the device having such rubber layers can have a total thickness of no more than 0.5 mm). For purposes of this application, the term “thin film” can mean a microscopically thin layer of material that is deposited onto a metal, ceramic, semiconductor or plastic base, or any device having such a component. Alternatively, for purposes of this application, it can also mean a component that is less than about 0.127 mm (0.005 inches) thick and contains a combination of conductive and dielectric layers or a device that has one or more such components, wherein the components can be combined in a stacked or layered configuration in the device. Finally, it is also understood, for purposes of this application, to have the definition that is understood by one of ordinary skill in the art.

Further, the various non-conducting thin-film components of the device 20 (and any other device embodiment herein) can be made of polyimide (“PI”), parylene C, liquid crystal polymer (“LCP”), or similar materials. Further, the conductive materials used in the device 20 (for the contacts and traces, for example) can be any one or more of platinum, platinum iridium, iridium oxide, titanium, or any other known conductive metal for use in spinal or neural probe devices.

In addition, the device 20 in the exemplary embodiment as shown in FIGS. 2A and 2B has a pusher receiving structure (or “body”) 24 attached or otherwise disposed at or near the distal end of the device 20. In one embodiment, the pusher receiving structure 24 can be a collar or nose 24 as shown that is configured to receive the distal end of a pusher device as discussed in additional detail below. That is, the collar 24 has a lumen 26 defined therethrough that allows for passage of a stylet 28 therethrough as shown (or any similar device such as a guidewire, for example) while being sized or structured such that a distal end of a pusher device (such as device 40 discussed below or any pusher embodiment disclosed or contemplated herein) can be positioned within the lumen 26 such that the pusher device engages with the structure 24 and makes it possible for the pusher to urge the structure 24 and entire device 20 in a desired direction as described herein. As such, the device 20 can be threaded onto or otherwise slidably attached to the stylet 28 by inserting the stylet 28 through the lumen 26 of the collar 24. In one embodiment, the collar 24 (and any pusher receiving structure embodiment disclosed or contemplated herein) can have an inner diameter ranging from about 0.018 inches to about 0.04 inches, a wall thickness ranging from about 0.005 inches to about 0.0008 inches, and a length ranging from about 0.04 inches to about 0.2 inches. Alternatively, the pusher receiving structure 24 can be any structure or body that can receive and engage with any pusher device embodiment as disclosed or contemplated herein (along with any stylet device or the like) and can have any known dimensions for such a device. According to certain specific implementations, the pusher receiving structure 24 can also be any of the pusher receiving structures (including the pockets, collars, flaps, or the like) as disclosed or contemplated in U.S. patent application Ser. No. 18/358,859, which is incorporated by reference above.

According to one embodiment, the stylet 28 (or any stylet embodiment disclosed or contemplated herein) can be a nitinol hypotube having a diameter of about 0.018 inches and containing both platinum and nitinol, wherein the platinum is disposed at the distal tip for visibility. Alternatively, the stylet 28 can be a nitinol wire having a diameter of about 0.035 inches, wherein the wire has platinum wound around the wire at various locations along the length for visibility. In this specific exemplary embodiment, the tip can be ground down or otherwise reduced in diameter to increase flexibility and steerability. Alternatively, the stylet 28 can be made of any metal material or combination thereof.

Any of the individual components, mechanisms, features, functionality, and/or dimensions of the device embodiment of FIGS. 2A and 2B described in detail above, including the pusher receiving structure 24, can be incorporated into any of the other system embodiments discussed below. Similarly, any of the individual components, mechanisms, features, functionality, and/or dimensions of any of the device embodiments described in detail below can be incorporated into the embodiment of FIGS. 2A and 2B or any of the other embodiments discussed below.

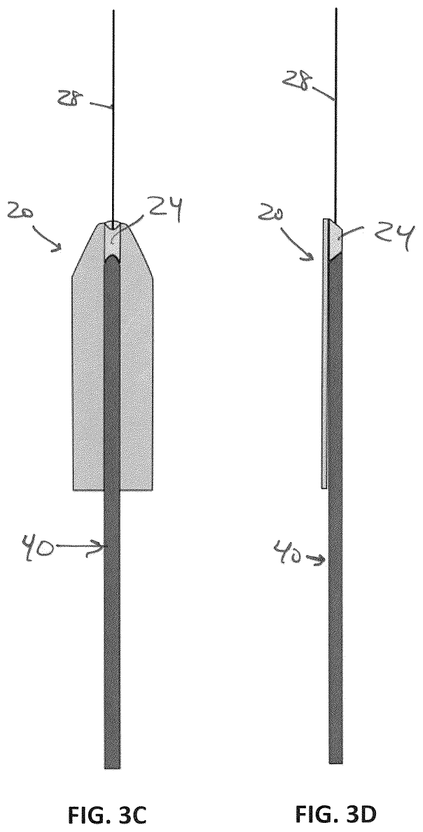

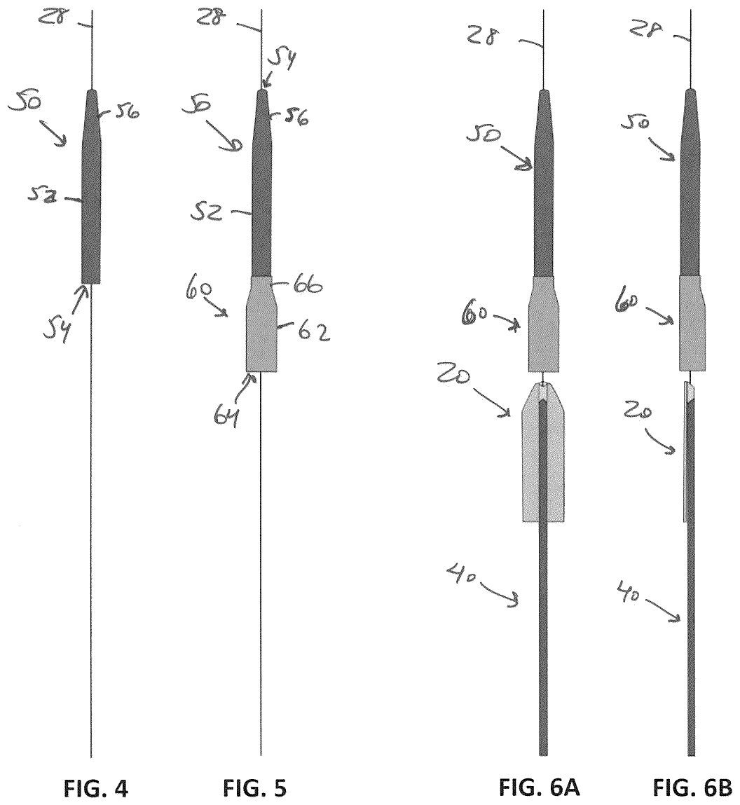

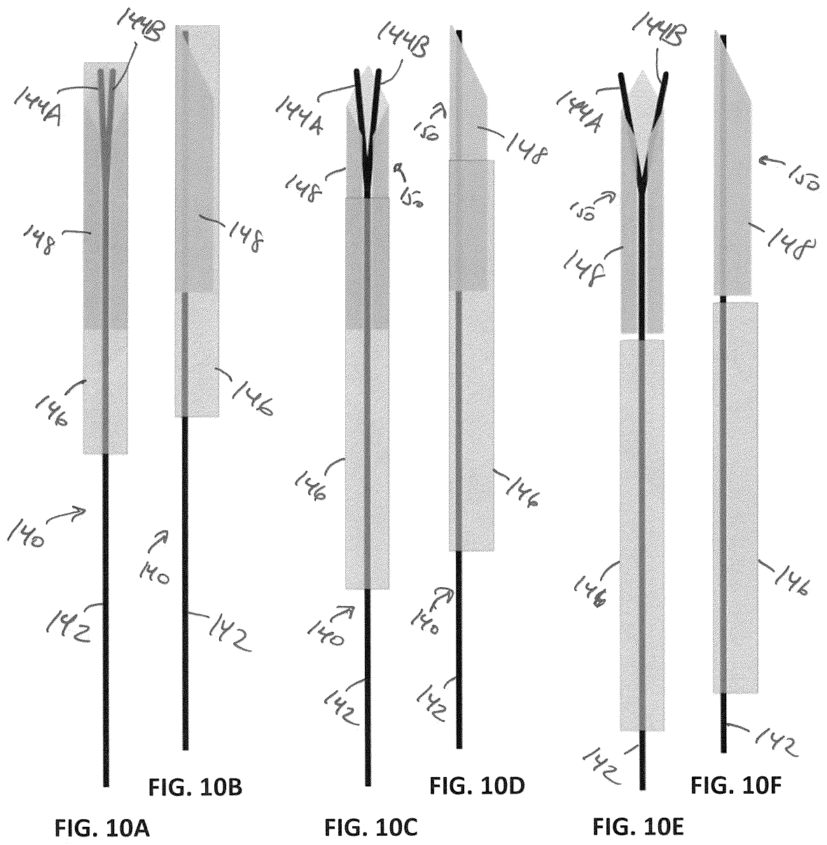

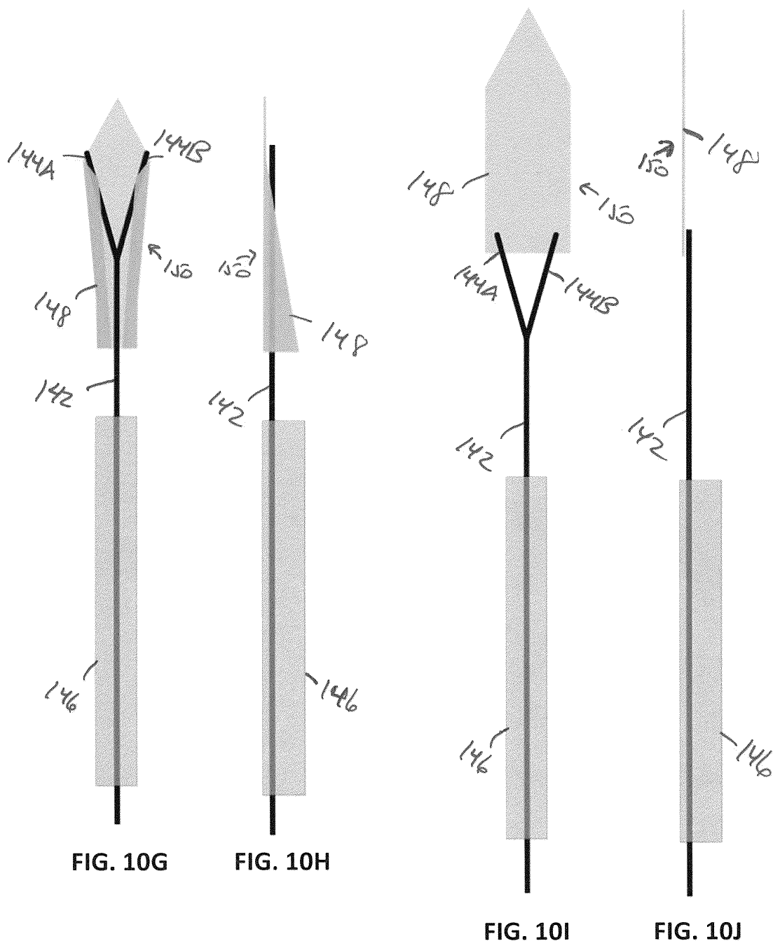

A pusher device 40 according to one implementation is depicted in FIGS. 3A-3D. The pusher device 40 is an elongate body 40 with a distal tip 42 that is configured to be coupleable with the collar 24 as shown. More specifically, the distal tip 42 can have any shape or configuration that allows for it to be coupleable with any of the various pusher receiving structures 24 disclosed or contemplated herein. For purposes of the exemplary implementation as shown in FIGS. 4A-4D, the distal tip 42 is a pointed tip 42 that can be positioned within the collar 24 such that the pusher 40 is attached to the collar 24 as shown in FIGS. 3C and 3D. Further, the pusher device 40 has a lumen (not shown) defined therethrough that allows the passage of any stylet (such as stylet 28) or any other such device (such as a guidewire, for example) therethrough, thereby allowing the pusher device 40 to be positioned over and slidable along such a stylet or guidewire. In one embodiment, the pusher device 40 (and any pusher embodiment disclosed or contemplated herein) has an inner diameter ranging from about 0.018 inches to about 0.04 inches, a wall thickness ranging from about 0.005 inches to about 0.0008 inches, and a length of about 15 inches or more. Alternatively, the pusher device 40 can be any of the various pusher device embodiments discussed in detail below in relation to FIGS. 10A-12D and can have any known dimensions for such a device. In use, the pusher device 40 can be positioned over the stylet 28 as shown in FIGS. 3A and 3B (or, more specifically, the proximal end of the stylet 28 is inserted into the lumen—not shown—at the distal end of the pusher device 40) and then advanced distally along the stylet 28 until the distal end 42 of the pusher device 40 engages with the collar 24 as shown in FIGS. 3C and 3D.

In accordance with certain stimulation device embodiments or implantation methods as shown in FIGS. 4 and 5, an introducer tool or “sheath” 50 can be used to assist with implantation. The sheath 50 can be any standard introducer sheath 50 for use in combination with a stylet 28 and various thin-film stimulation devices as disclosed or contemplated herein. In the exemplary embodiment as shown, the sheath 50 has a sheath body 52 with a lumen 54 defined therethrough and a tapered distal portion 56. According to some embodiments, the sheath 50 (and any sheath in any embodiment disclosed or contemplated herein) can have a size ranging from 5 French to about 10 French. Alternatively, the sheath 50 can be either a 7 French sheath or a 9 French sheath. In a further alternative, a smaller sheath 50 can be used when the stimulation device is a percutaneous device. In use, the sheath 50 can be inserted over a stylet 28 (or other similar device) prior to positioning the device as desired within the patient by inserting the stylet 28 through the lumen 54. Alternatively, the sheath 50 can first be positioned within the patient as desired and then the stylet 28 can be urged through the sheath 50.

According to certain optional embodiments as shown in FIG. 5, a funnel 60 can be used in combination with the sheath 50 for purposes of implantation of certain stimulation device embodiments. In one embodiment as shown, the funnel 60 has a funnel body 62 with a lumen 64 defined therethrough and a tapered distal portion 66 that can be coupled to or otherwise positioned in contact with or adjacent to the proximal end of the sheath 50 as shown. In use, the funnel 60 can be coupled to or otherwise positioned into contact with the proximal end of the sheath 50 such that the funnel 60 can be used to assist with insertion of a stimulation device therethrough, as will be described in additional details below. Once the funnel 60 is attached to the sheath 50, the sheath 50 and funnel 60 can be inserted over a stylet 28 (or other similar device) prior to positioning the device as desired within the patient by inserting the stylet 28 through the lumens 54, 64. Alternatively, the sheath 50 and funnel 60 can first be positioned within the patient as desired and then the stylet 28 can be urged through the funnel 60 and sheath 50. In a further alternative, the sheath 50 can be first positioned within the patient and then the funnel 60 can be attached thereto or otherwise position in contact with or adjacent to the sheath 50.

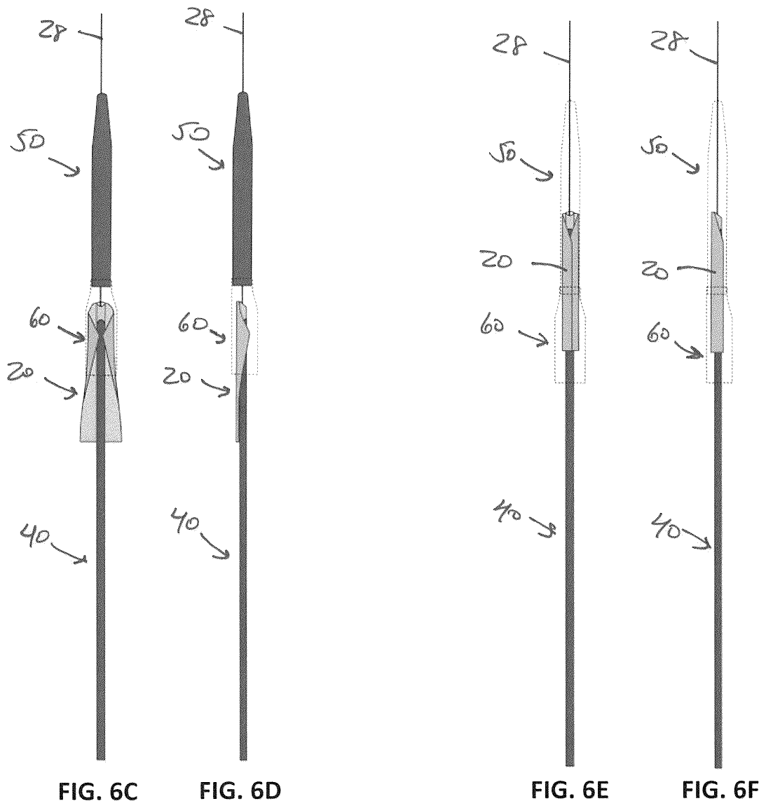

In use as shown in FIGS. 6A-6F, the deformable spinal cord stimulation device 20 as described above can be positioned over the stylet 28 as described elsewhere herein and urged toward the sheath 50 (and the funnel 60 in those embodiments in which the funnel 60 is used). Alternatively, any spinal cord stimulation device according to any embodiment disclosed or contemplated herein can be implanted using these devices and/or steps. According to some implementations, the stimulation device (such as device 20) is urged distally along the stylet 28 with the assistance of a pusher device 40 as shown. As best shown in FIGS. 6C and 6D, as the device (such as device 20) is urged into the funnel 60, the paddle 22 is urged into a folded or curled configuration by the funnel 60 such that the paddle 22 can fit through the lumen 66 of the funnel 60 as shown. Alternatively, the paddle 22 can be formed into any shape or configuration to fit through the funnel 60. In a further alternative in which the funnel 60 is not used, the paddle 22 can be formed into any shape or configuration to fit through the sheath 50. As best shown in FIGS. 6E and 6F, the paddle 22 remains in this curled, folded or otherwise reduced profile configuration as it passes through the sheath 50.

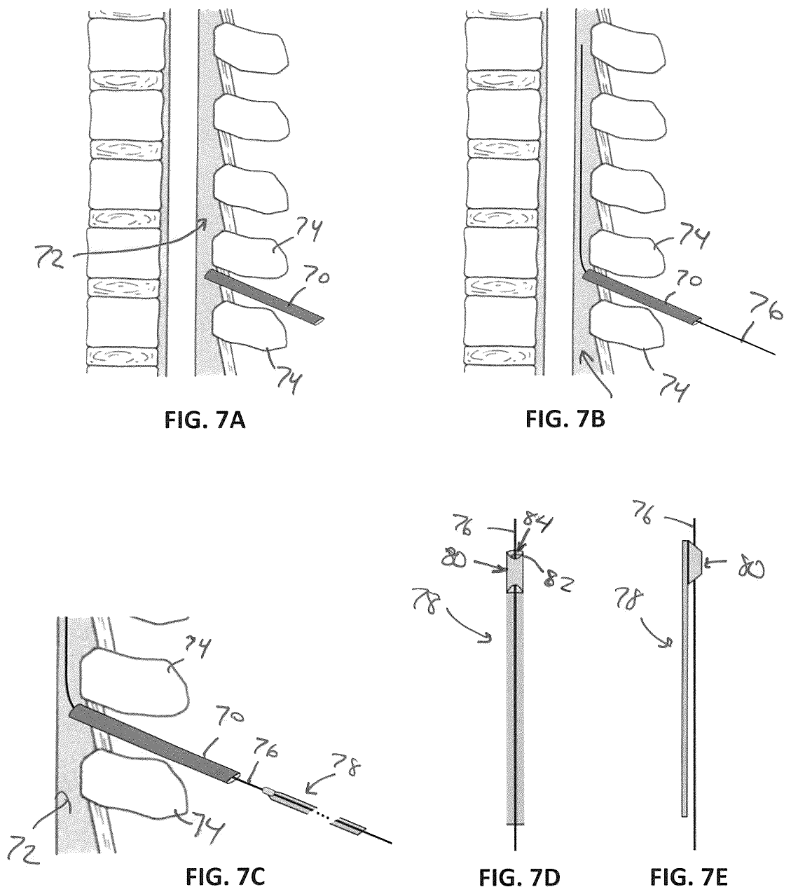

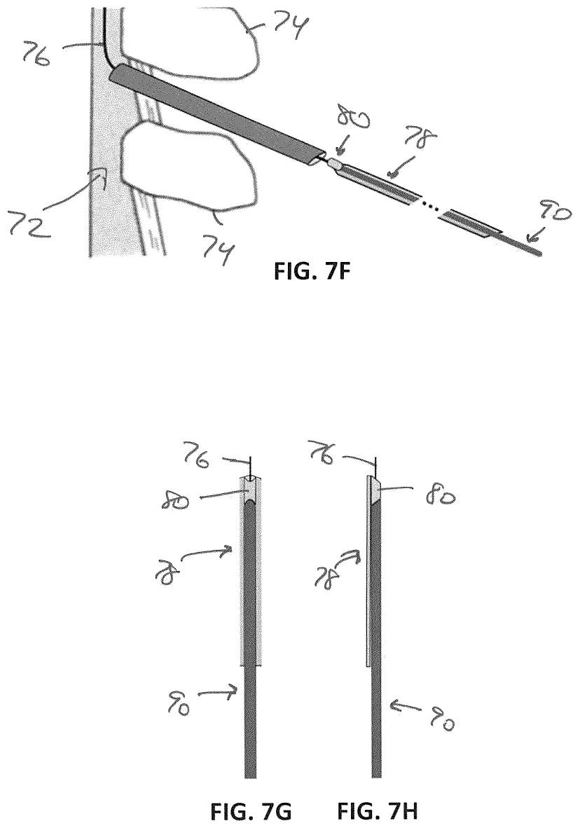

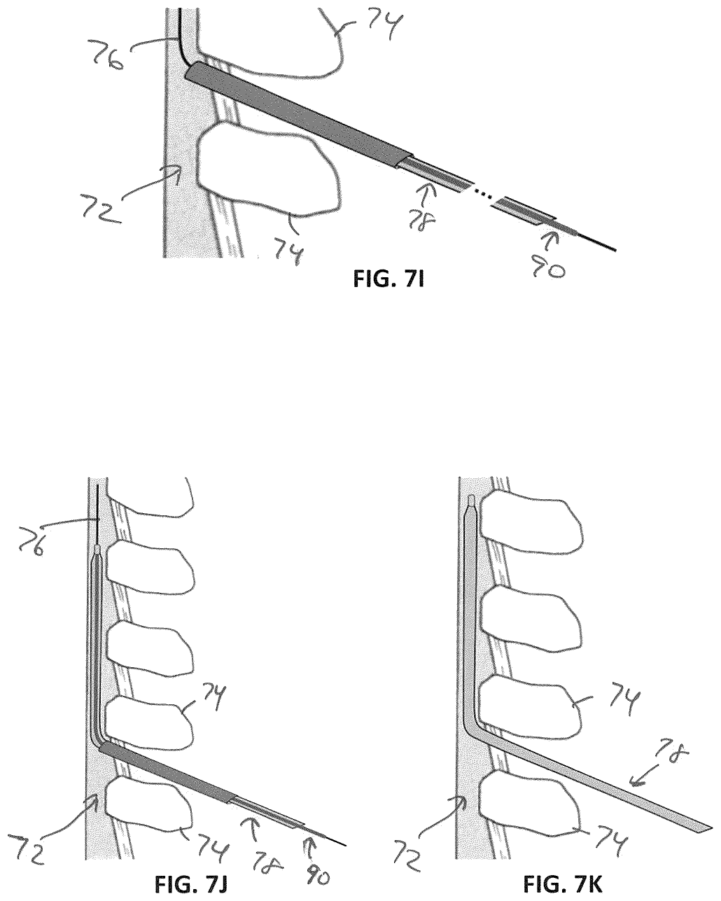

The various devices, tools, and/or steps discussed above can be used in the following manner according to certain implementations as shown in FIGS. 7A-9I in which the implantation methods disclosed or contemplated herein can include steering or directing any spinal cord stimulation device (such as device 20, for example) to the desired area adjacent to or in contact with the spinal cord via the proximal end (or “tail”) of the device. Such methods can include the use of devices or tools (such as the pusher receiving structures, pusher tools, sheaths, and/or funnels discussed above, for example) that can assist the surgeon to direct or position the device by grasping the proximal end of the tail to direct/position the device in the target area.

One embodiment of a method for implanting a temporary percutaneous stimulation device (such as, for example, the device 10 in FIG. 1A) is shown in FIGS. 7A-7K. According to this method, a needle 70 is first inserted into the epidural space 72 between two spinous processes 74 at a predetermined location along the length of the spinal column as shown in FIG. 7A. The needle 70 is inserted such that the distal end is disposed within the epidural space 72. According to one embodiment, the needle 70 user in this method (or any of the methods herein) can be a 14 AWG needle that is about 1.6 mm in diameter. Alternatively, the needle 70 can range in size from a 10 AWG needle to a 20 AWG needle. In a further alternative, the needle 70 can range in size from a 12 AWG to an 18 AWG needle. The needle 70 can have a length ranging from about 3 to about 7 inches. Alternatively, the needle 70 can have length ranging from about 4 to about 6 inches. In a further alternative, the needle 70 can be either about 4 inches long or about 6 inches long. In one specific example, the needle 70 is a Tuohy needle 70, which has a slight curve at the distal end to assist with directing the objects passing therethrough radially as they exit the needle. In certain implementations, the needle 70 is inserted without the assistance of any other device. Alternatively, any known tool or mechanism can be used to insert the needle 70. For example, in some embodiments, a larger needle can first be used to create an access point before inserting the needle 70. Alternatively, or in addition, in some exemplary cases, a small cut is formed in the skin at the target location for the needle with a scalpel or blade to ease insertion of the needle 70 therethrough.

The needle 70 is inserted along the length of the spinal column such that the needle 70 is inserted between two spinous processes 74 such that the distal end of the needle 70 is positioned within the epidural space 72 where the entire device 78 is inserted and ultimately where the proximal portion of the device 78 will be positioned, as will be described below (and depicted in the relevant figures).

Once the needle 70 has been inserted as desired, a stylet 76 is inserted through the needle 70 as best shown in FIG. 7B such that the stylet 76 extends distally through the epidural space 72 some predetermined distance from the distal end of the needle 70 and further extends proximally from the proximal end of the needle 70. In one embodiment, the stylet 76 is substantially similar to the stylet 28 discussed above. Alternatively, any known stylet 76 can be used.

At this point, as shown in FIG. 7C, a percutaneous stimulation device 78 is urged over the stylet 76 such that the stylet 76 is positioned through the pusher receiving structure 80 of the device 78. More specifically, the proximal end of the stylet 76 is inserted through the distal opening 82 of the lumen 84 in the pusher receiving structure 80 such that the device 78 can be advanced over the stylet 76. The percutaneous stimulation device 78 can be any such deformable thin-film stimulation device as disclosed or contemplated herein. Further, the pusher receiving structure 80 can be any such structure as disclosed or contemplated herein.

Once the device 78 is positioned over the stylet 76, a pusher device 90 is then urged over the stylet 76 and engaged with the pusher receiving structure 80 of the device 78 as shown in FIG. 2D. More specifically, the proximal end of the stylet 76 is inserted through the distal opening 92 of the pusher device 90 such that the pusher device 90 can be advanced over the stylet 76 and engaged with the pusher receiving structure 80 of the device 78. The pusher device 90 can be any of the various pusher devices disclosed or contemplated herein, including pusher device 40 or any of the pusher devices depicted in FIGS. 10A-12D and discussed in detail below.

Once the pusher 90 is engaged with the pusher receiving structure 80 of the stimulation device 78, the pusher 90 is used to advance the stimulation device 78 into the needle 70 as shown in FIG. 7E. More specifically, the surgeon urges the proximal end of the pusher 90 forward, thereby urging the pusher 90 and the stimulation device 78 distally into the needle 70. As best shown in FIG. 7F, as the surgeon continues to urge the pusher 90 forward, the stimulation device 78 is urged distally out of the needle 70 and along the stylet 76 in the epidural space 72 to the target location.

Once the stimulation device 78 is positioned as desired within the epidural space, the pusher 90, the stylet 76, and the needle 70 are removed, leaving only the stimulation device 78 as shown in FIG. 7G. More specifically, in certain embodiments, the devices are removed in the following order: the needle 70 is removed first, followed by the stylet 76, and then the pusher 90 is removed. Alternatively, the devices can be removed in any order.

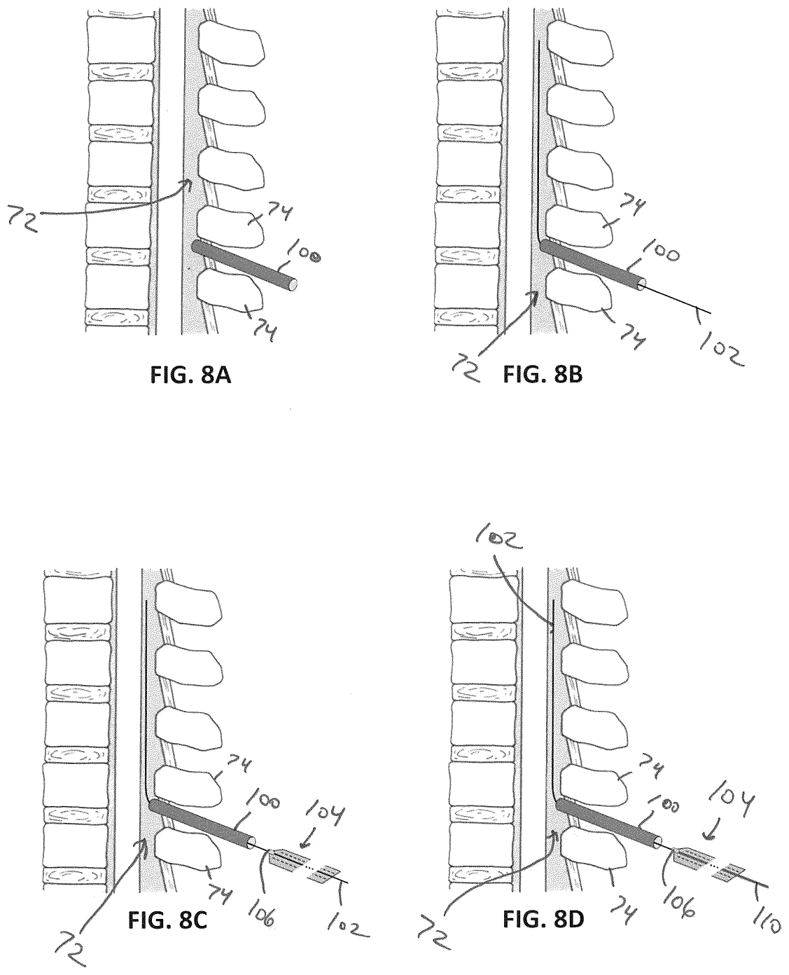

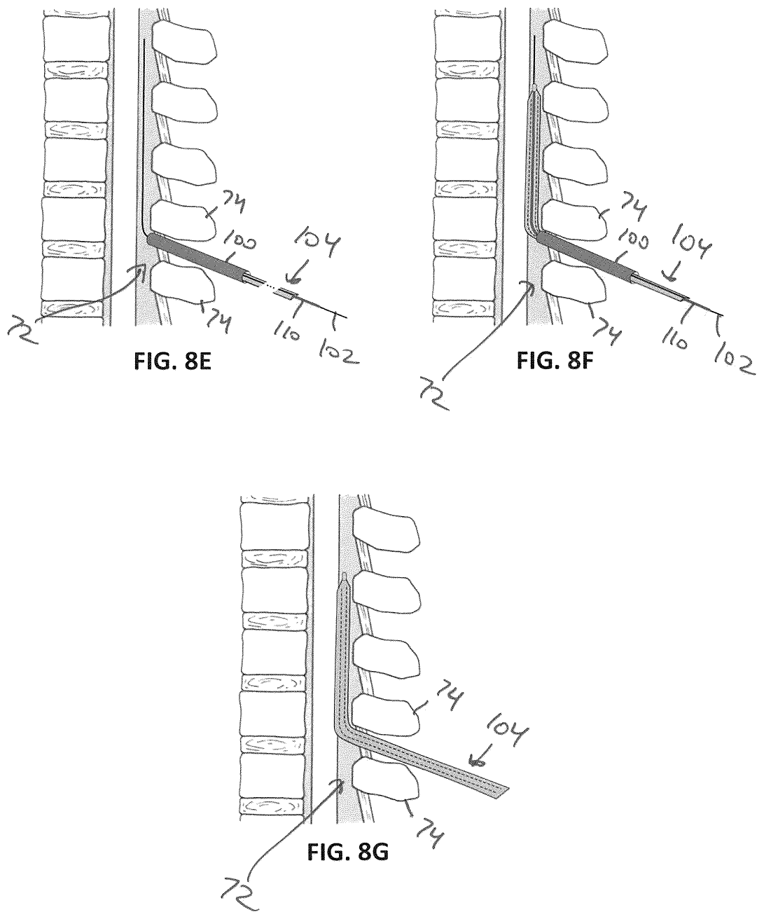

According to a further implementation, a method for implanting a permanent percutaneous stimulation device (such as, for example, the device 10 in FIG. 1A) is shown in FIGS. 8A-8G. According to this method, a needle 100 is first inserted into the epidural space 72 between two spinous processes 74 at a predetermined location along the length of the spinal column as shown in FIG. 8A. In one embodiment, the needle 100 is substantially similar to the needle 70 discussed above. The needle 100 is inserted such that the distal end is disposed within the epidural space 72. The needle 100 can have the same dimensions and features as the needle 70 as discussed above. In certain implementations, the needle 100 is inserted without the assistance of any other device. Alternatively, any known tool or mechanism can be used to insert the needle 100, including, for example, the larger needle or the small cut to the skin as discussed above with respect to the needle 70.

The needle 100 is inserted along the length of the spinal column such that the needle 100 is inserted between two spinous processes 74 such that the distal end of the needle 100 is positioned within the epidural space 72 where the entire device 104 is inserted and ultimately where the proximal portion of the device 104 will be positioned, as will be described below (and depicted in the relevant figures).

Once the needle 100 has been inserted as desired, a stylet 102 is inserted through the needle 100 as best shown in FIG. 8B such that the stylet 102 extends distally through the epidural space 72 some predetermined distance from the distal end of the needle 100 and further extends proximally from the proximal end of the needle 100. According to one embodiment, the stylet 102 can be substantially similar to the stylet 76 discussed above.

At this point, as shown in FIG. 8C, a percutaneous stimulation device 104 is urged over the stylet 102 such that the stylet 102 is positioned through the pusher receiving structure 106 of the device 104 in a fashion similar to that described above with respect to any of the stimulation device embodiments disclosed or contemplated herein, including devices 20, 78. The percutaneous stimulation device 104 has a pusher receiving structure 106 that is substantially similar to the pusher receiving structure embodiments 24, 80 discussed above or any other pusher receiving structure disclosed or contemplated herein. Further, according to certain embodiments, the percutaneous stimulation device 104 in this specific example can be any deformable device having deformable or thinned portions as disclosed or contemplated in U.S. patent application Ser. No. 18/358,859.

Once the device 104 is positioned over the stylet 102, a pusher device 110 is then urged over the stylet 102 and engaged with the pusher receiving structure 106 of the device 28 as shown in FIG. 8D. More specifically, the pusher device 110 can be any pusher device embodiment disclosed or contemplated herein, including pusher 40, 90 or any of the devices depicted in FIGS. 10A-12D and discussed in detail below and can be advanced over the stylet 102 and engaged with the pusher receiving structure 106 of the device 104 in a fashion similar to that described herein with respect to any of those pusher embodiments.

Once the pusher 110 is engaged with the pusher receiving structure 106 of the stimulation device 104, the pusher 110 is used to advance the stimulation device 104 into the needle 100 as shown in FIG. 8E. More specifically, the surgeon urges the proximal end of the pusher 110 forward, thereby urging the pusher 110 and the stimulation device 104 distally into the needle 100. In those embodiments in which the stimulation device 104 is the deformable stimulation device with the deformable or thinned portions as discussed above, as the device 104 is urged into the needle 100, the needle 100 causes the device 104 to fold like a leaflet or otherwise deform along its two thinned portions.

As best shown in FIG. 8F, as the surgeon continues to urge the pusher 110 forward, the stimulation device 104 is urged distally out of the needle 100 and along the stylet 102 in the epidural space 72 to the target location. Referring back to those exemplary embodiments in which the deformable device with deformable or thinned portions is used, the device body deploys (expands from the folded or deformed position) back into its expanded or normal configuration as it exits the needle 100.

Once the stimulation device 104 is positioned as desired within the epidural space 72, the pusher 110, the stylet 102, and the needle 100 are removed, leaving only the stimulation device 104 as shown in FIG. 8G. More specifically, in certain embodiments, the devices are removed in the following order: the needle 100 is removed first, followed by the stylet 106, and then the pusher 110 is removed. Alternatively, the devices can be removed in any order.

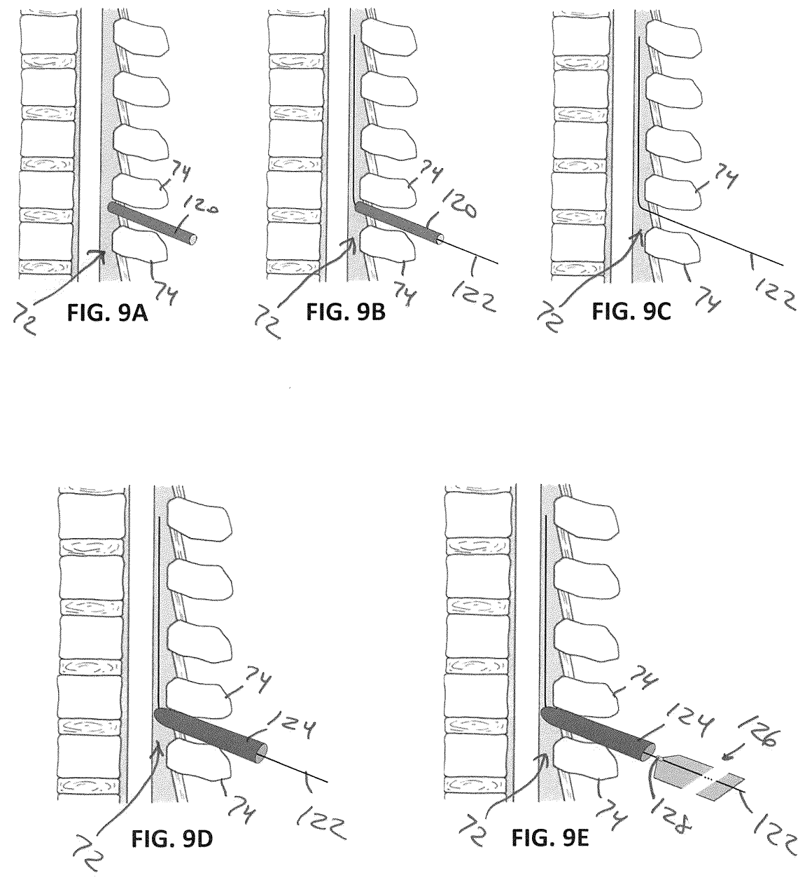

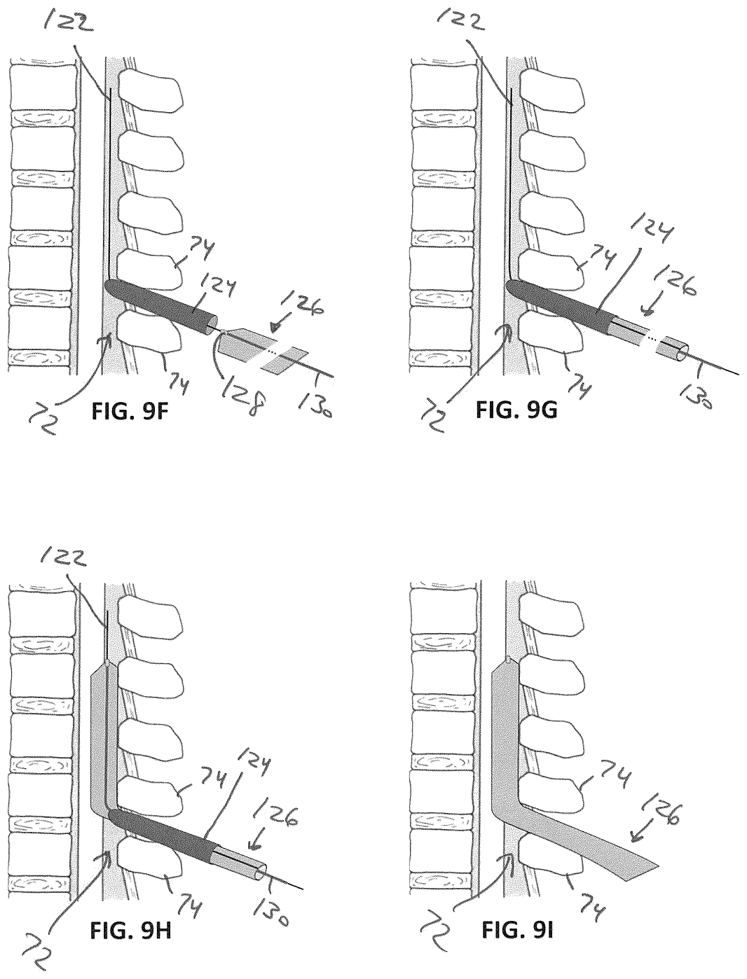

According to yet another embodiment, a method for implanting a paddle stimulation device (such as, for example, the device 12 in FIG. 1B) is shown in FIG. 9A. According to this method, a needle 120 is first inserted into the epidural space 72 between two spinous processes 74 at a predetermined location along the length of the spinal column as shown in FIG. 9A. In one embodiment, the needle 120 is substantially similar to either of the needle embodiments 70, 100 discussed above and can have the same dimensions and features as those needles 70, 100. The needle 120 is inserted such that the distal end is disposed within the epidural space 72. In certain implementations, the needle 120 is inserted without the assistance of any other device. Alternatively, any known tool or mechanism can be used to insert the needle 120, including, for example, the larger needle or the small cut to the skin as discussed above with respect to the needle 70.

The needle 120 is inserted along the length of the spinal column such that the needle 120 is inserted between two spinous processes 74 such that the distal end of the needle 120 is positioned within the epidural space 72 where the entire device 126 is inserted and ultimately where the proximal portion of the device 126 will be positioned, as will be described below (and depicted in the relevant figures).

Once the needle 120 has been inserted as desired, a stylet 122 is inserted through the needle 120 as best shown in FIG. 9B such that the stylet 122 extends distally through the epidural space 72 some predetermined distance from the distal end of the needle 120 and further extends proximally from the proximal end of the needle 120. According to one embodiment, the stylet 122 can be substantially similar to either of the stylets 76, 102 discussed above.

Once the stylet 122 is positioned as desired, the needle 120 is removed as shown in FIG. 9C, thereby leaving solely the stylet 122 in place. At this point, a sheath 124 is inserted over the stylet 122 and into the epidural space 72 between two spinous processes 74 as shown in FIG. 9D. In one embodiment, the sheath 124 is substantially similar to the sheath embodiment 50 discussed above. Alternatively, the sheath 124 can be any introducer device as disclosed or contemplated herein. The sheath 124 is inserted such that the distal end is disposed within the epidural space 72 as shown.

At this point, as shown in FIG. 9E, a paddle stimulation device 126 is urged over the stylet 122 such that the stylet 122 is positioned through the pusher receiving structure 128 of the device 126 in a fashion similar to that described above with respect to any of the stimulation device embodiments disclosed or contemplated herein, including devices 20, 78, 104. The paddle stimulation device 126 has a pusher receiving structure 128 that is substantially similar to the pusher receiving structure embodiments 24, 80, 106 discussed above or any other pusher receiving structure disclosed or contemplated herein. Further, according to certain embodiments, the percutaneous stimulation device 126 in this specific example can be any deformable device having deformable or thinned portions as disclosed or contemplated in U.S. patent application Ser. No. 18/358,859.

Once the device 126 is positioned over the stylet 122, a pusher device 130 is then urged over the stylet 122 and engaged with the pusher receiving structure 128 of the device 126 as shown in FIG. 9F. More specifically, the pusher device 130 can be any pusher device embodiment disclosed or contemplated herein, including pusher 40, 90, 110 or any of the devices depicted in FIGS. 10A-12D and discussed in detail below and can be advanced over the stylet 122 and engaged with the pusher receiving structure 128 of the device 126 in a fashion similar to that described herein with respect to any of those pusher embodiments.

Once the pusher 130 is engaged with the pusher receiving structure 128 of the stimulation device 126, the pusher 130 is used to advance the stimulation device 126 into the sheath 124 as shown in FIG. 9G. More specifically, the surgeon urges the proximal end of the pusher 130 forward, thereby urging the pusher 130 and the stimulation device 126 distally into the sheath 124. In those embodiments in which the stimulation device 126 is the deformable stimulation device with the deformable or thinned portions as discussed above, as the device 126 is urged into the sheath 124, the needle 120 causes the device 126 to fold like a leaflet or otherwise deform along its two thinned portions. Alternatively, the device 126 can curl into a tube as shown in FIGS. 9G and 9H.

According to certain implementations, a funnel such as the funnel 60 can also be used in combination with the sheath 124 in a fashion similar to that depicted in FIGS. 6A-6F and discussed in detail above. Alternatively, the implantation can be performed without a funnel.

As best shown in FIG. 9H, as the surgeon continues to urge the pusher 130 forward, the stimulation device 126 is urged distally out of the sheath 124 and along the stylet 122 in the epidural space 72 to the target location. Further, the device 126 unfurls as it exits the sheath 124 as shown in FIG. 9H. For example, referring back to those exemplary embodiments in which the deformable device with deformable or thinned portions is used, the device body deploys (expands from the folded or deformed position) back into its expanded or normal configuration as it exits the sheath 124. Alternatively, in certain implementations, any of the pusher devices as depicted below in FIGS. 10A-12D can be used as described below to urge the device 126 back into its expanded or normal configuration after exit from the sheath 124.

Once the stimulation device 126 is positioned as desired within the epidural space 72, the pusher 130, the stylet 122, and the sheath 124 are removed, leaving only the stimulation device 126 as shown in FIG. 9I. More specifically, in certain embodiments, the devices are removed in the following order: the sheath 124 is removed first, followed by the stylet 122, and then the pusher 130 is removed. Alternatively, the devices can be removed in any order.

In another implementation, any of the devices, tools, and or components as depicted in FIGS. 6A-6F and the various steps as discussed in detail above can be incorporated into any of the devices and/or steps as described above with respect to FIGS. 9A-9I.

As mentioned above, certain pusher devices as disclosed or contemplated herein can not only be used to urge any of the various spinal cord stimulation devices herein into the desired position adjacent to the spinal cord, but also to help deploy any deployable electrode array section (such as a paddle) in any such stimulation device. Alternatively, certain of the deployment devices herein can serve only as deployment devices and not as pusher devices. That is, the various methods disclosed or contemplated herein can also help to ensure the unfurling and/or full deployment of spinal cord stimulation devices with paddle arrays (such as the exemplary device 12 depicted in FIG. 1B, for example). In standard, known implantation methods, the wide paddle leads can be deployed via minimally-invasive introduction devices that require the wide paddle leads to be curled or otherwise contracted during implantation. One problem with such methods is that there can be difficulties unfurling or otherwise expanding the paddle back to its fully-deployed configuration once the paddle has been positioned within the patient. Use of the various methods and devices herein can help to ensure that the paddle is expanded to its fully deployed configuration.

One exemplary pusher and deployment device 140 and related method of deploying a stimulation device with a paddle is depicted in FIGS. 10A-10J. It is noted that the stimulation device is first implanted according to any of the various implantation method embodiments disclosed or contemplated herein. Further, according to one embodiment, once the stimulation device is urged out of the needle or sheath or other introduction device into the desired position on or adjacent to the spinal cord, the following steps can be taken to utilize the deployment device 140 to ensure the paddle of the device is fully unfurled/deployed. As shown, the deployment device 140 has an elongate body 142 and expandable deployment wings 144A, 144B that move between a retracted position (as shown in FIG. 10A) and a deployed position (as shown FIGS. 10G and 10I). In the retracted position, the wings 144A, 144B are compressed and tensioned, while the wings 144A, 144B are in their untensioned state in the deployed position. Thus, the wings 144A, 144B are configured to expand to their deployed position when external forces are removed.

According to certain embodiments, the pusher and deployment device 140 can be used according to the following steps. In some exemplary embodiments in which the device 140 is also used as a pusher 140, the device is coupled to the paddle 148 of the device 150 according to any of the various methods for engaging a pusher with a stimulation device as disclosed or contemplated elsewhere herein. Thus, as shown in FIGS. 10A and 10B, the pusher and deployment device 140 can be used to urge the stimulation device 150 into and through the needle (or sheath in some embodiments) 146 as discussed elsewhere with the retracted wings 144A, 144B disposed within the curled paddle 148. As the pusher/deployment device 140 urges the stimulation device 150 out of the needle/sheath 146 as best shown in FIGS. 10C-10D, the wings 144A, 144B are released from the needle/sheath 146 and thus can move toward their deployed position. As the wings 144A, 144B begin to move toward their deployed positions as best shown in FIGS. 10E and 10F, the wings 144A, 144B begin to urge the curled up paddle 148 into its deployed position. Thus, as best shown in FIGS. 10G and 10H, when the wings 144A-B ultimately reach their deployed position, the distal portion of the paddle 148 that the wings 144A-B are disposed in has been urged into its deployed position, while the proximal portions are still somewhat curled up as shown. At this point, the surgeon can begin to retract or otherwise urge the deployment device 140 proximally such that the wings 144A, 144B move proximally along the paddle 148, thereby urging the remaining curled portions into their deployed position as best shown in FIGS. 101 and 10J.

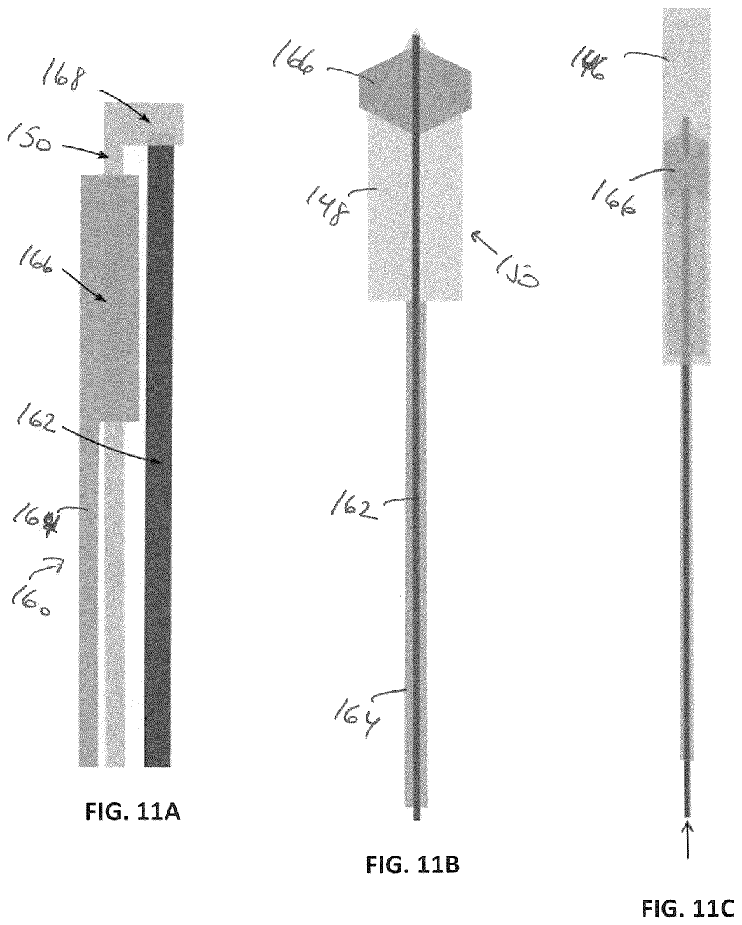

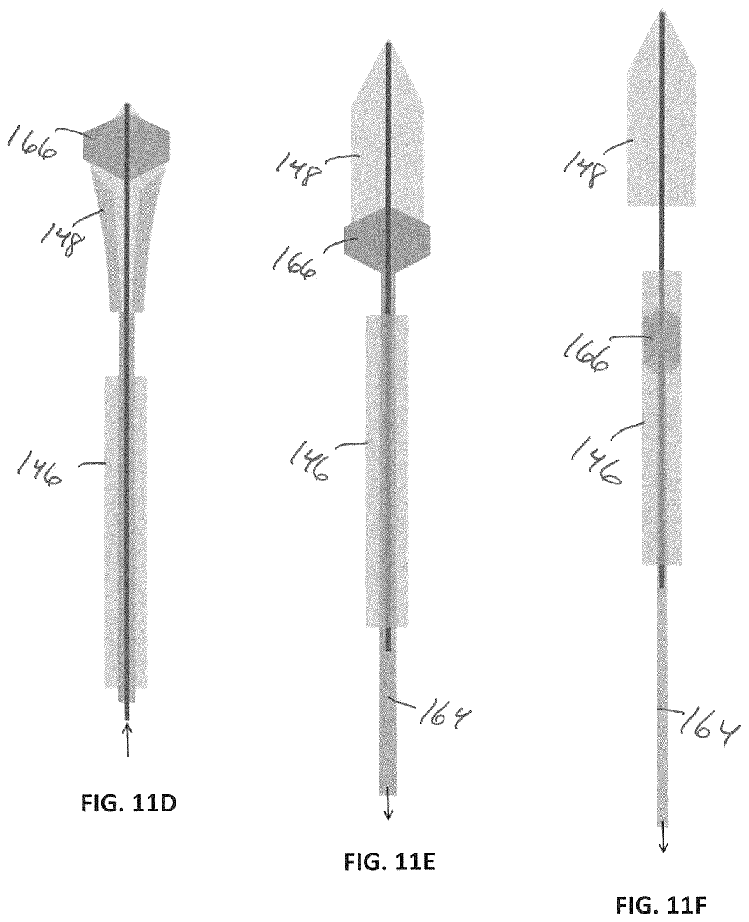

Another exemplary deployment device 160 and related method of deploying a stimulation device 150 with a paddle 148 is depicted in FIGS. 11A-11F. In this implementation, the device 160 and the pusher 162 are separate devices as shown, wherein the deployment device 160 has an elongate body 164 and an expandable sleeve 166 that can be disposed within the curled paddle 148 during insertion of the device 150 according to any of the method embodiments herein. In one embodiment, the expandable sleeve 166 is an expandable body 166 that is made of nitinol or any other known shape memory material and can move between a retracted position (as shown in FIGS. 11C and 11F) and a deployed position (as shown FIGS. 11B, 11D, and 11E). In the retracted position, the expandable body 166 is compressed and tensioned, while the body 166 is in its untensioned state in the deployed position. Thus, the expandable sleeve 166 is configured to expand to their deployed position when external forces are removed.

According to certain embodiments, the deployment device 160 can be used according to the following steps. In some exemplary embodiments in which the pusher 162 is a separate device, the pusher 162 and the deployment device 160 are both urged distally into the needle (or sheath) 146 with the stimulation device 150 as shown in FIG. 11C with the pusher 162 coupled to the stimulation device 150 via a pusher receiving structure 168 according to any of the embodiments disclosed or contemplated herein and further with the expandable sleeve 166 in its retracted or compressed position within the curled paddle 148 as shown. As the deployment device 160 and the stimulation device 150 are urged out of the needle/sheath 146 as best shown in FIG. 11D, the expandable sleeve 166 is released from the needle/sheath 146 and thus moves into its deployed position, thereby urging the distal end of the curled up paddle 148 into its deployed position (while the proximal portions are still somewhat curled up as shown). At this point, the surgeon can begin to retract or otherwise urge the deployment device 160 proximally such that the deployed sleeve 166 moves proximally along the paddle 148, thereby urging the remaining curled portions into their deployed position as best shown in FIG. 11E. The deployment device 160 can then be retracted through the needle (or sheath) 146 such that it moves back into its retracted or compressed position as shown in FIG. 11F and ultimately is retracted out of the patient.

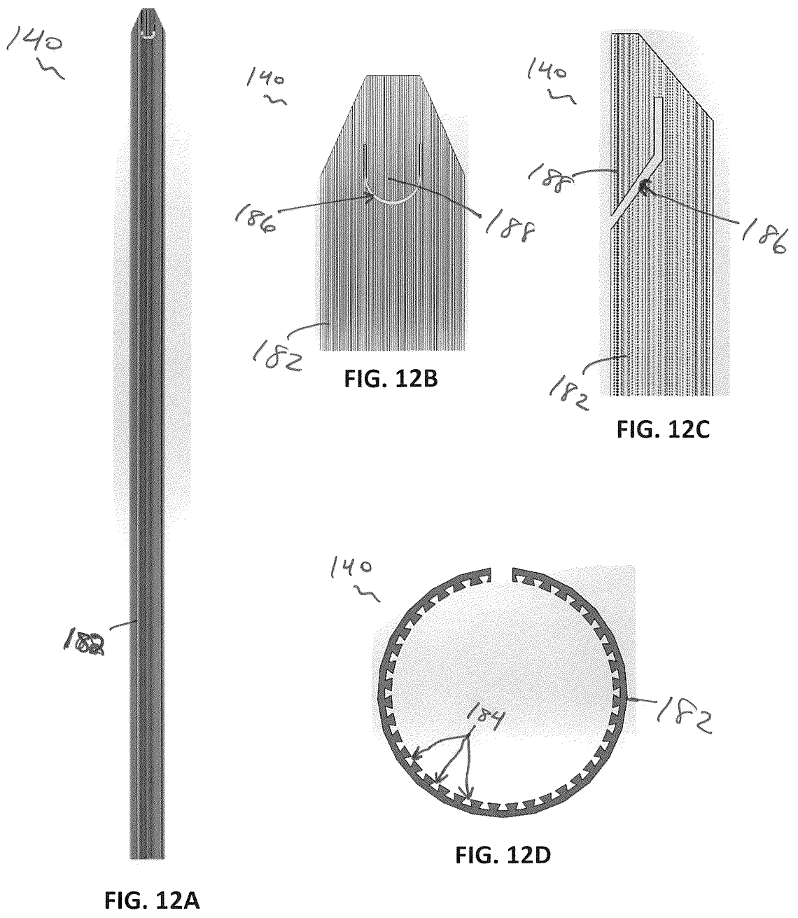

Another combination pusher and deployment device 180 according to a further implementation is depicted in FIGS. 12A-12D. This exemplary pusher and deployment device 140 has an elongate body 182 having a series of channels or trenches 184 defined in one side of the body 182 such that the body 182 can be curled into a tubular shape as shown in FIG. 12D via the channels 184. Further, the device 140 has a slit 186 defined in the body 182 near the distal end thereof. In embodiment, the slit 186 is a curved or U-shaped slit 186 that forms a flap 188 as shown. According to one embodiment, the pusher and deployment device 140 can be coupled to the stimulation device (not shown) to be implanted via the slit 186. In other words, the stimulation device can be urged into the slit 186 such that the stimulation device is disposed between the flap 188 and the rest of the device body 182.

Once the stimulation device (not shown) is attached to the pusher device 180 via the slit 186 and flap 188, the pusher 180 is curled into its tubular shape as best shown in FIG. 12D, thereby having the stimulation device (not shown) disposed within the lumen 190 formed in the pusher 180 in its tubular shape. At this point, the pusher 180 and stimulation device contained therein can be inserted through a cannula, sheath, or needle and thereby implanted into the target area on or adjacent to the spinal cord of the patient according to any of the various implantation method embodiments disclosed or contemplated herein. Once the distal end of the pusher 180 (and the stimulation device contained therein) is urged out of the needle or sheath or other introduction device into the desired position on or adjacent to the spinal cord, the body 182 of the deployment device 140 moves back into its unfurled position, thereby urging the stimulation device (not shown) into its unfurled position as well. Once the stimulation device is fully unfurled/deployed, the pusher device 180 can be uncoupled from the stimulation device and retracted, thereby leaving the stimulation device in place as desired.

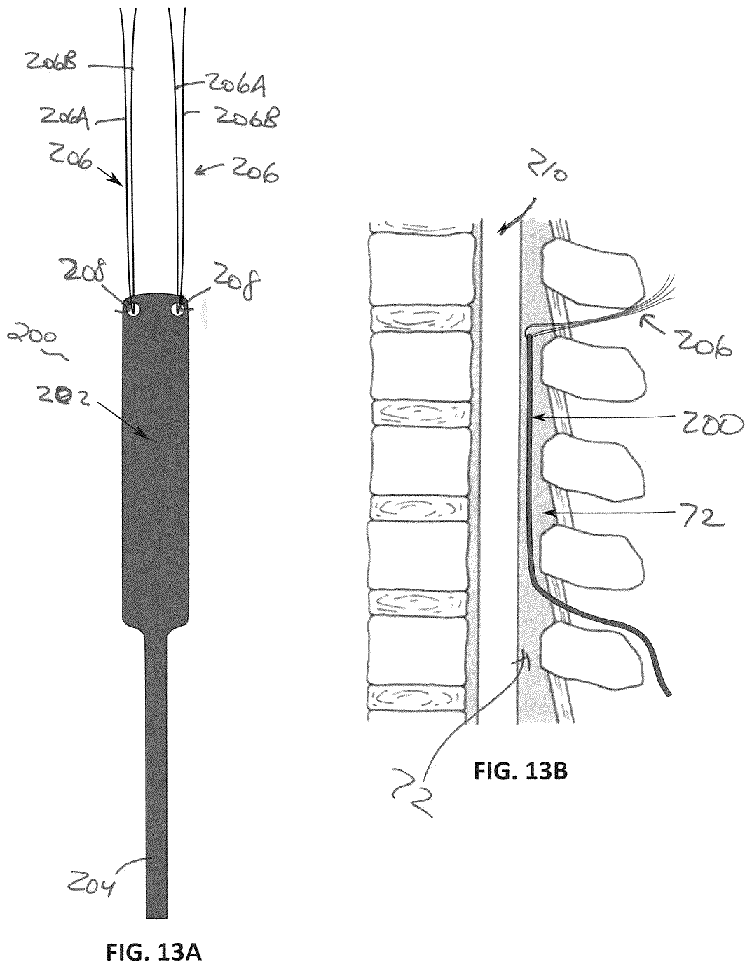

Alternatively, various devices and methods are provided herein that provide for steering, directing, and/or positioning spinal cord stimulation devices via both ends of the such devices. More specifically, as depicted in FIGS. 13A and 13B, certain implantation method embodiments disclosed or contemplated herein can include steering or directing any spinal cord stimulation device 200 at both ends of the device 200. Such methods can include the use of sutures 206 that are attached to a distal portion (a portion of the contact array 202, typically) of the exemplary stimulation device 200 such that a surgeon can direct or position the device 200 by grasping the proximal end of the tail 204 to direct/position the proximal portion of the device 200 and further by grasping the sutures 206 to direct/position the distal portion of the device 200. In certain implementations such as the exemplary implementation as shown, the sutures 206 are attached to the device 200 by positioning the sutures 206 through openings or holes 208 formed in the device 200 as shown. Alternatively, any attachment device, mechanism, or method can be used. The sutures 206 used in this exemplary method and in any of the various method embodiments herein can be Prolene sutures 206, or, alternatively, can be any other known sterile surgical sutures. In this implementation, one suture 206 is passed through each opening 208 such that two lengths 206A, 206B extend from the opening 208 as shown. Thus, as shown in FIG. 13B and as will be described in further detail below, once the device 200 is disposed within the epidural space 72 adjacent to the spinal cord 210, the sutures 206 extend out of the epidural space 72 as shown such that they are accessible to the surgeon. Hence, the various implantation methods disclosed or contemplated herein allow the surgeon (or other user) to control the position of the stimulation device 200 throughout the procedure and ensure that the device 200 is properly positioned in relation to the spinal cord 210.

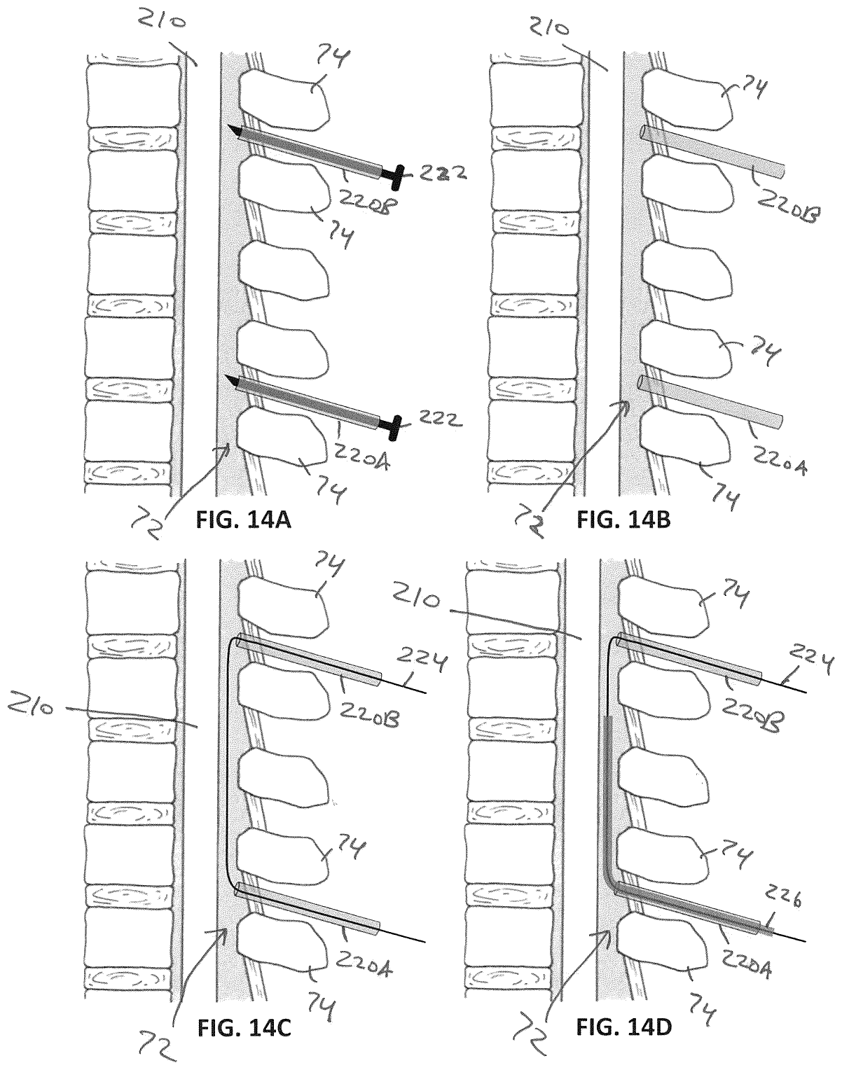

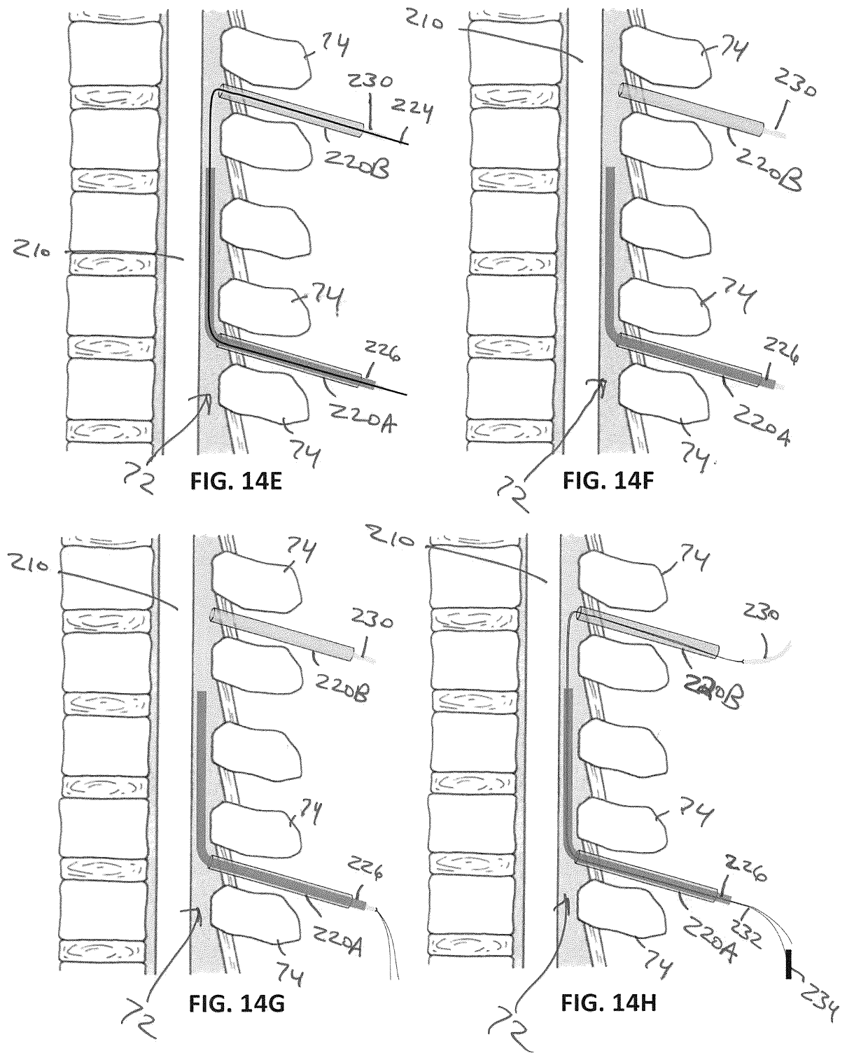

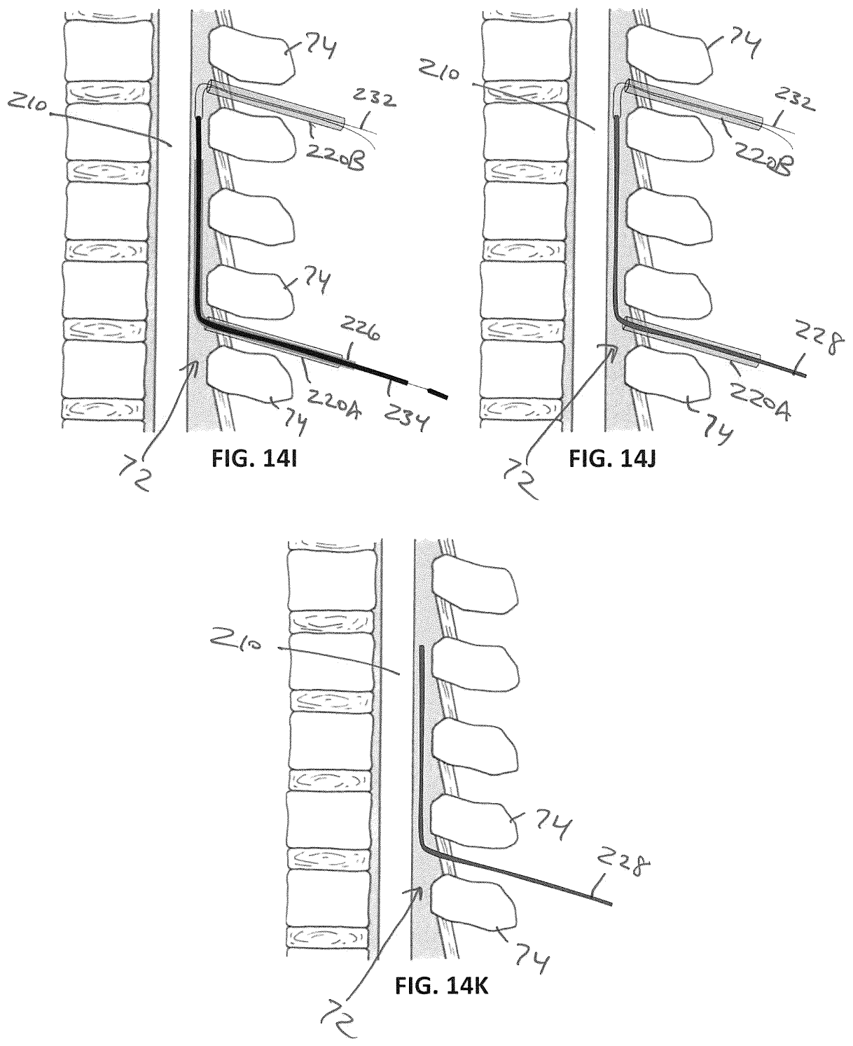

One embodiment of a method for implanting a percutaneous stimulation device (such as, for example, the device 10 in FIG. 1A) is shown in FIGS. 14A-14K. According to this method, catheter tubes 220A, 220B (or “sheaths”) are first inserted into the epidural space 72 between two spinous processes 74 at two different locations along the length of the spinal column as shown in FIGS. 14A and 14B. The tubes 220A, 220B are inserted such that the distal ends of each are disposed within the epidural space 72. In certain implementations, the tubes 220A, 220B are inserted using trocar catheter needles 222 as best shown in FIG. 14A, after which the needles 222 are removed and the tubes 220A, 220B remain, as best shown in FIG. 14B. Alternatively, any known tool or mechanism can be used to insert the two catheter tubes 220A, 220B.

The tubes 220A, 220B are inserted along the length of the spinal column such that the first tube 220A is inserted between two spinous processes 74 such that the distal end of the tube 220A is positioned within the epidural space 72 where the proximal portion of the device 228 will be positioned, while the second tube 220B is inserted between two spinous processes 74 spaced from the other two processes 74 such that the distal end of the tube 220B is positioned within the epidural space 72 where the distal portion of the device 228 will be positioned, as will be described below (and depicted in the relevant figures).

Once the tubes 220A, 220B have been inserted as desired and the needles 222 (or other insertion tools) have been removed, a guidewire 224 is inserted through both catheter tubes 220A, 220B such that the guidewire 224 extends through the epidural space 72 between the distal ends of the tubes 220A, 220B, as best shown in FIG. 14C. The guidewire 224 can be inserted first through either of the tubes 220A, 220B, such that the distal end of the guidewire 224 can first be inserted through the tube 220A, then urged through the epidural space 72, into the distal end of the tube 220B and through the tube 220B such that the guidewire 224 extends out of the proximal end of the tube 220B. Alternatively, the guidewire 224 can be inserted in the reverse direction (first through the tube 220B, through the epidural space 72, and then through the tube 220A). Once the guidewire 224 has been inserted (regardless of direction), the guidewire 224 should be positioned through both tubes 220A, 220B and the epidural space 72 such that the guidewire 224 extends out of the proximal ends of both tubes 220A, 220B as best shown in FIG. 14C. In certain embodiments, certain known tools can be used to urge the guidewire 224 through the tubes 220A, 220B. For example, in one implementation, forceps can be used to pull the distal end of the guidewire 224 from the exit catheter tube (whether tube 220A or tube 220B).

At this point, as shown in FIG. 14D, a lead introduction device 226 is urged over the guidewire 224 and through the catheter tube 220A until the distal end of the introduction device 226 is disposed at the appropriate location within the epidural space 72 for delivery of the stimulation device 228. In certain embodiments, the introduction device 226 is an introducer sheath 226. Alternatively, any known introduction device for use in inserting a stimulation device into a target area can be used.

Once the introduction device 226 is positioned as desired, a suture introduction device 230 is inserted over the guidewire 224 and through the lead introduction device 230, as best shown in FIG. 14E. More specifically, in one embodiment, the introduction device 230 is inserted over the guidewire 224 and through the tube 220B via the proximal opening of the tube 220B, into the introduction device 226, and through the tube 220A via the distal opening of the tube 220A such that the distal end of the introduction device 230 extends out of the proximal end of the introduction device 226 as shown.

In the exemplary embodiment as shown in FIG. 14E, the suture introduction device 230 is a catheter 230 with an outer diameter that is less than the inner diameter of the introduction device 226. Alternatively, the suture introduction device 230 can be any known introduction device for use in urging sutures 232 through the lead introduction device 226 and the tubes 220A, 220B. Further, in certain embodiments, the suture introduction device 230 has at least one small hole (not shown) defined in the distal end of the device 230 to which the sutures 232 can be attached (as discussed below). Alternatively, the suture introduction device 230 can have any known attachment component, structure, or mechanism at or near its distal end that allows for attachment of the sutures 232 to the device 230.

Once the suture introduction device 230 is positioned such that the distal end extends out of the proximal end of the lead introduction device 226, the guidewire 224 is removed, as best shown in FIG. 14F. And once the guidewire 224 is removed, the sutures 232 can be coupled to the distal end of the device 230 as shown in FIG. 14G. Alternatively, the sutures 232 are coupled to a proximal end of the device 230 before the device 230 is inserted.

Once the sutures 232 are attached to the suture introduction device 230, the introduction device 230 is retracted proximally at its proximal end such that the distal end (with the sutures 232 attached) is pulled through the introduction device 226 and tube 220A and then through tube 220B such that the introduction device 230 is fully retracted from the tubes 220A, 220B as shown in FIG. 14H. At this point, the sutures 232 are positioned in both tubes 220A, 220B and extend through the epidural space 72 as shown. As also shown in the figure, the proximal end of the sutures 232 have the stimulation device 228 attached thereto, with the device 228 disposed within a stimulation device delivery case 234 as shown. That is, the sutures 232 are attached to the device 228 in a fashion similar to that described above with respect to FIG. 13A.

The device delivery case 234 is an enclosure or housing that can house the stimulation device 228 therein. It is understood that the case 234 can be any known housing or enclosure for such purposes. The sutures 232 are attached to the device 228 within the case 234 such that the sutures 232 extend out of the case 234 (through holes in the case 234 or the like) and thus remain attached to the device 228 when the case 234 is removed as described below.

At this point, the suture introduction device 230 is detached from the sutures 232. The sutures 232 are then urged distally out of the proximal end of the tube 220B such that the sutures 232 pull the case 234 (with the stimulation device 228 disposed therein) distally through the tube 220A and into the epidural space 72 to the desired position as shown in FIG. 14I. In certain embodiments, a stylet or other similar tool can be used to help advance the case 234 (and device 228 therein) to the desired position within the epidural space 72.

Once the device 228 is disposed within the epidural space 72 as desired, the device case 234 and the introduction device 226 are removed proximally from the tube 220A as shown in FIG. 14J, leaving the device 228 disposed in the desired position. At this point, the device 228 position can be adjusted as needed using the proximal tail of the device 228 and the sutures 232 attached to the distal end of the device 228.

Once the device 228 is positioned/repositioned, the tubes 220A, 220B and the sutures 232 are removed as shown in FIG. 14K. In some implementations, the sutures 232 are detached from the distal end of the device 228 as shown. Alternatively, the sutures 232 can be cut off where they extend out of the patient.

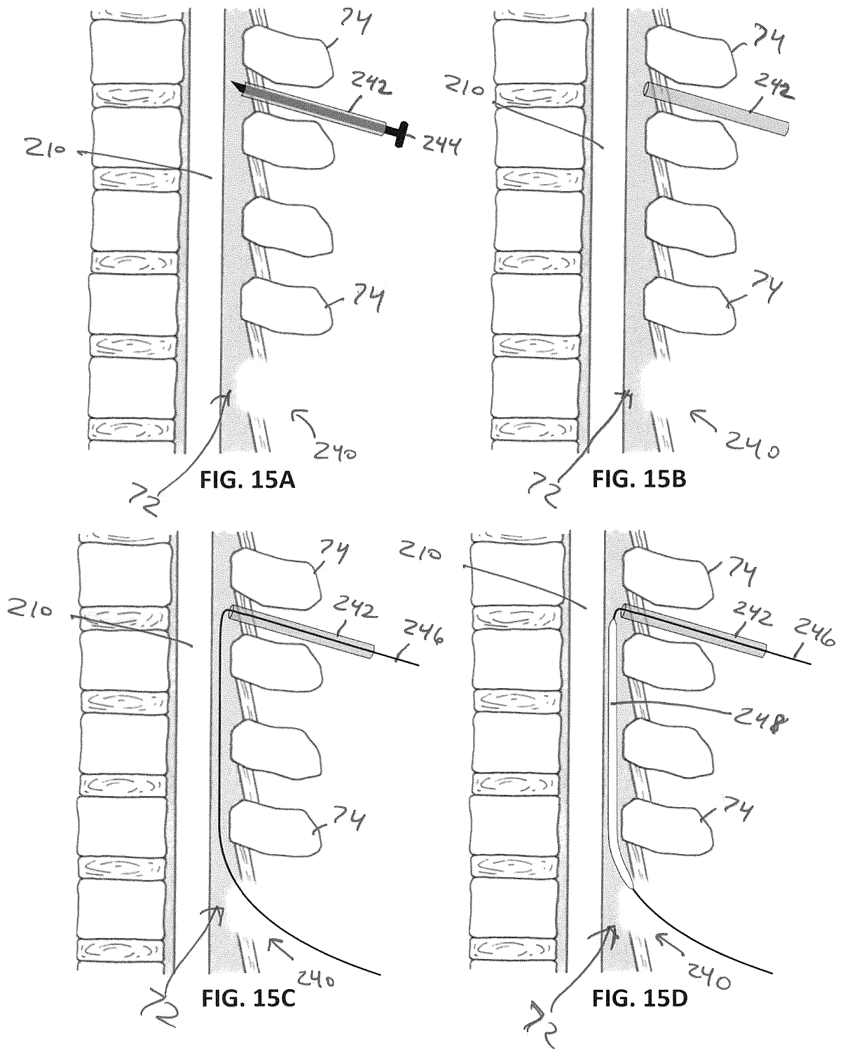

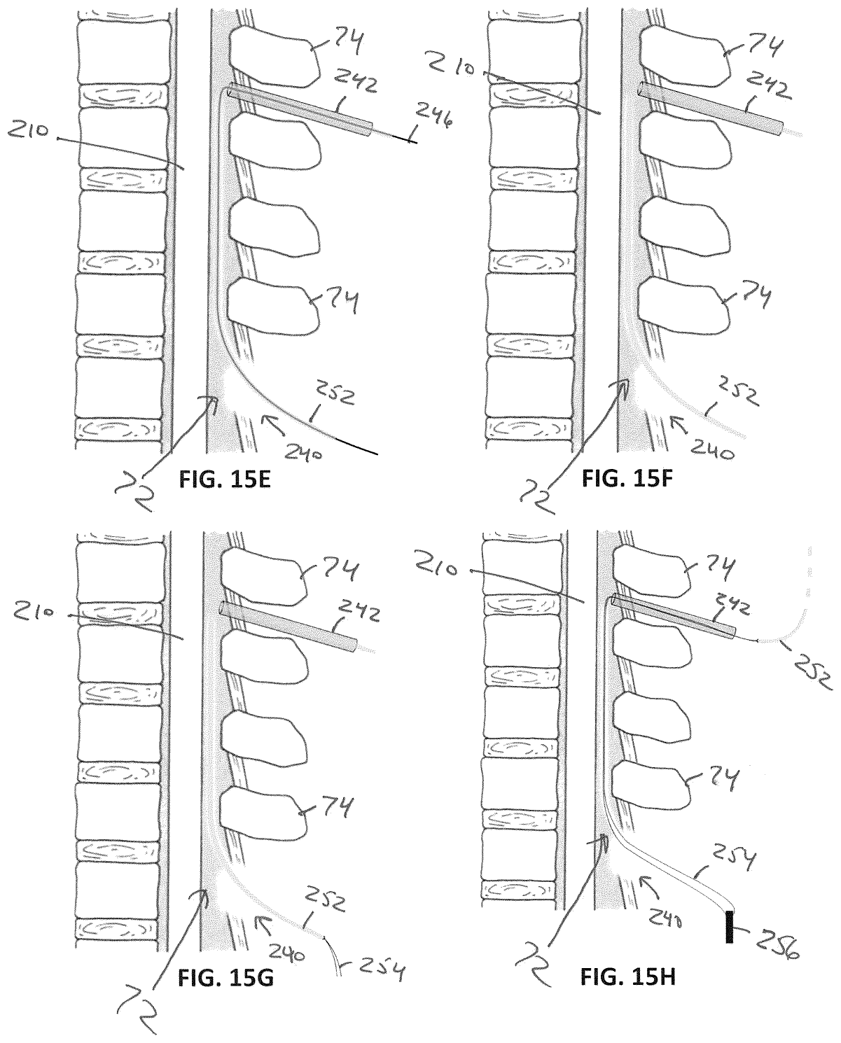

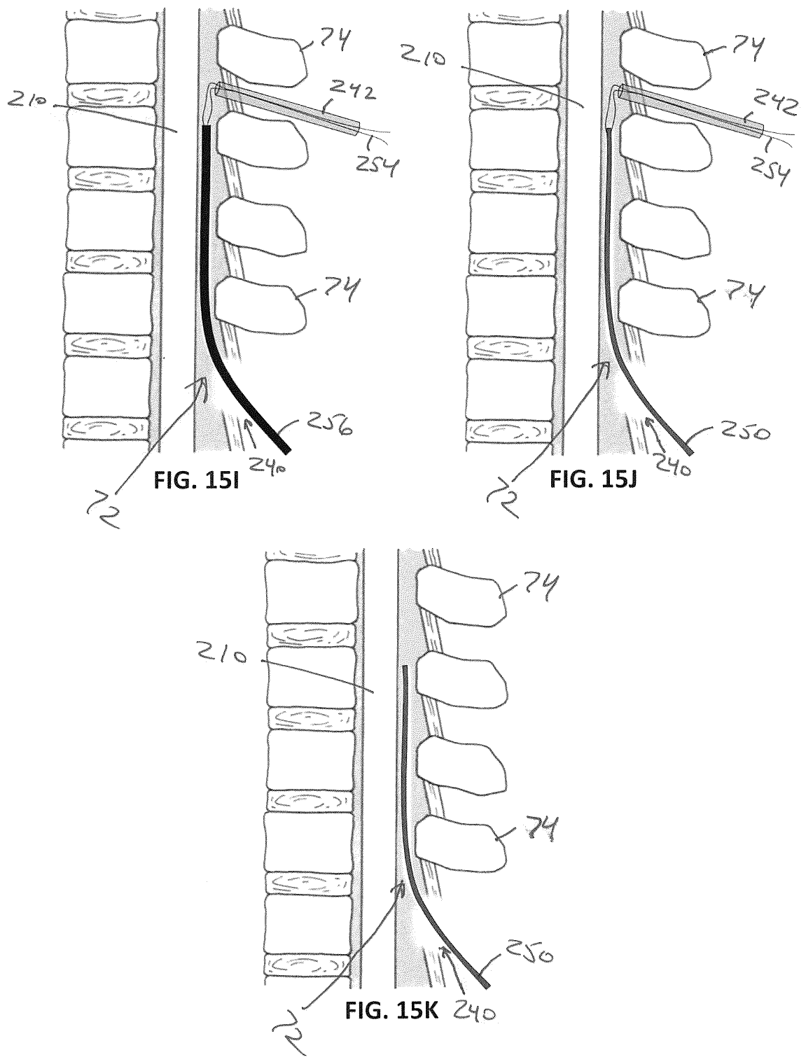

One embodiment of a method for implanting a paddle stimulation device (such as, for example, the device 12 in FIG. 1B) is shown in FIGS. 15A-15K. According to this method, a standard laminotomy can first be performed to remove one of the spinous processes, thereby resulting in an opening 240 where the spinous process had been. Once the laminotomy is complete, one catheter tube 242 (or “sheath”) is first inserted into the epidural space 72 between two spinous processes 74 at a predetermined location in relation to the opening 240 along the length of the spinal column as shown in FIGS. 15A and 15B. The tube 242 is inserted such that the distal end is disposed within the epidural space 72. In certain implementations, the tube 242 is inserted using a trocar catheter needle 244 as best shown in FIG. 15A, after which the needle 244 is removed and the tube 242 remains, as best shown in FIG. 15B. Alternatively, any known tool or mechanism can be used to insert the catheter tube 242.

The tube 242 is inserted along the length of the spinal column between the two spinous processes 74 as shown such that the distal end of the tube 242 is positioned within the epidural space 72 where the distal portion of the device 250 will be positioned and further such that the proximal end of the device 250 will be positioned adjacent to the opening 240, as will be described below (and depicted in the relevant figures).

Once the tube 242 has been inserted as desired and the needle 244 (or other insertion tool) has been removed, a guidewire 246 is inserted through the catheter tube 242 such that the guidewire 246 extends through the catheter tube 242, through the epidural space 72 between the distal end of the tube 242, and out through the opening 240, as best shown in FIG. 15C. The guidewire 246 can be inserted first through the tube 242 such that the distal end of the guidewire 246 can first be inserted through the tube 242, then urged through the epidural space 72, and out of the opening 240 such that the guidewire 246 extends out of the opening 240. Alternatively, the guidewire 246 can be inserted in the reverse direction (first through the opening 240, through the epidural space 72, and then through the tube 242). Once the guidewire 246 has been inserted (regardless of direction), the guidewire 246 should be positioned through the tube 242, the epidural space 72, and the opening 240 such that the guidewire 246 extends out of the tube 242 at one end and the opening 240 at the other as best shown in FIG. 15C. In certain embodiments, certain known tools can be used to urge the guidewire 246 into position. For example, in one implementation, forceps can be used to pull the distal end of the guidewire 246 from the catheter tube 242 or the opening 240.

Once the guidewire has been positioned as desired, an optional dilation step can be performed, as shown in FIG. 15D. More specifically, a dilator 248 can be urged over the guidewire 246 and through the opening 240 into the epidural space 72 to form a pocket in the epidural space 72 to receive the stimulation device 250 and then is retracted. In certain embodiments, a series of dilators 248 of increasing size can be used to ensure that the pocket formed in the epidural space 72 is large enough to receive the device 250. Alternatively, the dilation step can be performed at any earlier stage in the process. In a further alternative implementation, no dilation step is performed.

At this point, as shown in FIG. 15E, a suture introduction device 252 is inserted over the guidewire 246. More specifically, in one embodiment, the introduction device 252 is inserted over the guidewire 246 and through the tube 242, through the epidural space 72, and out of the opening 240 such that the distal end of the introduction device 252 extends out of the opening 240 as shown.

In the exemplary embodiment as shown in FIG. 15E, the suture introduction device 252 is a catheter 252 with an outer diameter that is less than the inner diameter of the tube 242. Alternatively, the suture introduction device 252 can be any known introduction device for use in urging sutures 254 through into position as described herein. Further, in certain embodiments, the suture introduction device 252 has at least one small hole (not shown) defined in the distal end of the device 252 to which the sutures 254 can be attached (as discussed below). Alternatively, the suture introduction device 252 can have any known attachment component, structure, or mechanism at or near its distal end that allows for attachment of the sutures 254 to the device 252.

Once the suture introduction device 252 is positioned such that the distal end extends out of the opening 240, the guidewire 246 is removed, as best shown in FIG. 15F. And once the guidewire 246 is removed, the sutures 254 can be coupled to the distal end of device 252 as shown in FIG. 15G. Alternatively, the sutures 254 are coupled to a proximal end of the device 252 before the device 252 is inserted.

At this point, the introduction device 252 is retracted proximally at its proximal end such that the distal end (with the sutures 254 attached) is pulled through the opening 240, the epidural space 72, and then through tube 242 such that the introduction device 252 is fully retracted from the tube 242 as shown in FIG. 15H. At this point, the sutures 254 are positioned in the opening 240, the tube 242, and through the epidural space 72 as shown. As also shown in the figure, the proximal end of the sutures 254 have the stimulation device 250 attached thereto, with the device 250 disposed within a stimulation device delivery case 256 as shown. That is, the sutures 254 are attached to the device 250 in a fashion similar to that described above with respect to FIG. 13A.

The device delivery case 256 is substantially similar to the case 234 discussed above with respect to FIGS. 14A-14K.

At this point, the suture introduction device 252 is detached from the sutures 254. The sutures 254 are then urged distally out of the proximal end of the tube 242 such that the sutures 254 pull the case 256 (with the stimulation device 250 disposed therein) distally through the opening 240 and into the epidural space 72 to the desired position as shown in FIG. 15I. In certain embodiments, a stylet or other similar tool can be used to help advance the case 256 (and device 250 therein) to the desired position within the epidural space 72.

Once the device 250 is disposed within the epidural space 72 as desired, the device case 256 is removed proximally through the opening 240 as shown in FIG. 15J, leaving the device 250 disposed in the desired position. At this point, the device 250 position can be adjusted as needed using the proximal tail of the device 250 and the sutures 254 attached to the distal end of the device 250.

Once the device 250 is positioned/repositioned, the tube 242 and the sutures 254 are removed as shown in FIG. 15K. In some implementations, the sutures 254 are detached from the distal end of the device 250 as shown. Alternatively, the sutures 254 can be cut off where they extend out of the patient.

The various method embodiments herein can be performed using stimulation devices (such as devices 20 or 250, for example) that are opaque or have visual indicators visible via various medical visualization technologies, including X-ray fluoroscopy or any other known technology.

Further, in accordance with certain implementations, any of the methods disclosed or contemplated herein can also include an additional step after implantation in which the sutures 254 are kept in place (remain attached to the distal portion of the stimulation device) such that the device can be anchored in place at each end of the device during the tissue healing phase after implantation (a period ranging from about 1 week to about 2 weeks). That is, in any of the methods herein, the implanted position of the device is maintained during the tissue healing phase by anchoring the device in place via the sutures 254 at the distal end and the lead at the proximal end for about two weeks after implantation. Alternatively, the device is anchored via the sutures 254 and the lead for a time period ranging from about 1 week to about 3 weeks. In a further embodiment, the device anchored via the sutures 254 and the lead for any known period of time that is about equal to the tissue healing phase. This step can help to ensure that the device stays in about the desired position and prevents or mitigates the migration of the device after implantation—a problem that was identified in a recent clinical study. In one specific embodiment, the device does not migrate more than about 6 mm as a result of the anchoring. Alternatively, the device does not migrate more than about 4 mm. In a further embodiment, the device does not migrate more than about 2 mm. In yet another implementation, the device does not migrate as a result of the anchoring.

While the various systems described above are separate implementations, any of the individual components, mechanisms, or devices, and related features and functionality, within the various system embodiments described in detail above can be incorporated into any of the other system embodiments herein.

The terms “about” and “substantially,” as used herein, refers to variation that can occur (including in numerical quantity or structure), for example, through typical measuring techniques and equipment, with respect to any quantifiable variable, including, but not limited to, mass, volume, time, distance, wave length, frequency, voltage, current, and electromagnetic field. Further, there is certain inadvertent error and variation in the real world that is likely through differences in the manufacture, source, or precision of the components used to make the various components or carry out the methods and the like. The terms “about” and “substantially” also encompass these variations. The term “about” and “substantially” can include any variation of 5% or 10%, or any amount—including any integer—between 0% and 10%. Further, whether or not modified by the term “about” or “substantially,” the claims include equivalents to the quantities or amounts.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾ This applies regardless of the breadth of the range. Although the various embodiments have been described with reference to preferred implementations, persons skilled in the art will recognize that changes may be made in form and detail without departing from the spirit and scope thereof.

Although the various embodiments have been described with reference to preferred implementations, persons skilled in the art will recognize that changes may be made in form and detail without departing from the spirit and scope thereof.

What is claimed is:

1. A thin film spinal cord stimulation system comprising:
(a) a spinal cord stimulation device comprising:
(i) an elongate thin film lead body; and
(ii) a thin film electrode body disposed at one end of the elongate thin film lead body, the thin film electrode body comprising:
(A) at least two contacts disposed on the electrode body; and
(B) a pusher receiving structure disposed on a distal portion of the thin film electrode body; and
(b) an elongate pusher device comprising:
(i) an elongate pusher body; and
(ii) a distal tip disposed at a distal end of the elongate pusher body, wherein the distal tip is constructed and arranged to be coupleable with the pusher receiving structure.

2. The thin film spinal cord stimulation system of claim 1, further comprising an introduction needle comprising a lumen sized to receive the spinal cord stimulation device and the elongate pusher device therethrough.

3. The thin film spinal cord stimulation system of claim 2, further comprising a steerable stylet configured to be positionable through the introduction needle.

4. The thin film spinal cord stimulation system of claim 2, further comprising an introduction sheath comprising a lumen having a larger inner diameter than the lumen of the introduction needle.

5. The thin film spinal cord stimulation system of claim 4, further comprising a funnel comprising a lumen and a distal end, wherein the distal end is coupleable to a proximal end of the introduction sheath.

6. The thin film spinal cord stimulation system of claim 1, wherein the pusher receiving structure is a collar comprising a lumen.

7. The thin film spinal cord stimulation system of claim 1, further comprising a deployment device comprising:
(a) an elongate deployment device body; and
(b) an expandable structure disposed at a distal end of the elongate deployment device body, the expandable structure configured to move between a retracted configuration and an expanded configuration.

8. The thin film spinal cord stimulation system of claim 7, wherein the expandable structure comprises two expandable wings or an expandable sleeve.

9. A method of implanting a thin film spinal cord stimulation device, the method comprising:
inserting an introduction needle between two spinous processes and into an epidural space of a patient;
inserting a steerable stylet through the introduction needle such that the guidewire extends distally from the introduction needle within the epidural space;
inserting the thin film spinal cord stimulation device over the steerable stylet;
inserting a pusher device over the steerable stylet and into engaging contact with the thin film spinal cord stimulation device;
urging the thin film spinal cord stimulation device through the introduction needle by urging the pusher device distally over the steerable stylet such that the thin film spinal cord stimulation device is disposed in the epidural space; and
removing the pusher device, steerable stylet, and introduction needle.

10. The method of claim 9, wherein the thin film spinal cord stimulation device comprises:
(a) an elongate thin film lead body; and
(b) a thin film electrode body disposed at one end of the elongate thin film lead body, the thin film electrode body comprising:
(i) at least two contacts disposed on the electrode body; and
(i) a pusher receiving structure disposed on a distal portion of the thin film electrode body.

11. The method of claim 10, wherein the inserting the pusher device into engaging contact with the thin film spinal cord stimulation device comprises urging the pusher device into contact with the pusher receiving structure such that the pusher device is coupled with the pusher receiving structure.

12. The method of claim 9, wherein the removing the introduction needle comprises removing the introduction needle after inserting the steerable stylet through the introduction needle.

13. The method of claim 12, further comprising inserting an introduction sheath over the steerable stylet after removing the introduction needle such that the introduction sheath is inserted between the two spinous processes and into the epidural space.

14. The method of claim 13, further comprising inserting a funnel over the steerable stylet and coupling the funnel to the introduction sheath.

15. The method of claim 13, further comprising inserting a deployment device through the introduction sheath and retracting the deployment device to assist with the deployment of the thin film spinal cord stimulation device.

16. A method of implanting a thin film spinal cord stimulation device, the method comprising:
inserting an introduction needle between two spinous processes and into an epidural space of a patient;
inserting a steerable stylet through the introduction needle such that the guidewire extends distally from the introduction needle within the epidural space;
inserting the thin film spinal cord stimulation device over the steerable stylet, wherein the thin film spinal cord stimulation device comprises:
(a) an elongate thin film lead body; and
(b) a thin film electrode body disposed at one end of the elongate thin film lead body, the thin film electrode body comprising:
(i) at least two contacts disposed on the electrode body; and
(ii) a pusher receiving structure disposed on a distal portion of the thin film electrode body;
inserting a pusher device over the steerable stylet and coupling the pusher device with the pusher receiving structure;
urging the thin film spinal cord stimulation device through the introduction needle by urging the pusher device distally over the steerable stylet such that the thin film spinal cord stimulation device is disposed in the epidural space; and
removing the pusher device, steerable stylet, and introduction needle.

17. The method of claim 16, wherein the inserting the thin film spinal cord stimulation device over the steerable stylet comprises inserting a proximal end of the steerable stylet through the pusher receiving structure.

18. The method of claim 16, further comprises:
removing the introduction needle after inserting the steerable stylet through the introduction needle; and
inserting an introduction sheath over the steerable stylet such that the introduction sheath is inserted between the two spinous processes and into the epidural space.

19. The method of claim 18, further comprising inserting a funnel over the steerable stylet and coupling the funnel to the introduction sheath.

20. The method of claim 18, further comprising inserting a deployment device through the introduction sheath and retracting the deployment device to assist with the deployment of the thin film spinal cord stimulation device.
